(19) United States
(12) Patent Application Publication
(10) Pub. No.: US 2018/0110747 A1
(43) Pub. Date: Apr. 26, 2018

(54) USE OF THIA OXO COMPOUNDS FOR LOWERING APO C3

(71) Applicant: Pronova Biopharma Norge AS, Osio (NO)

(72) Inventor: David Alan FRASER, Blommenholm (NO)

(21) Appl. No.: 15/562,554

(22) PCT Filed: Apr. 1, 2015
(86) PCT No.: PCT/IB2015/001316

§ 371 Date: Sep. 28, 2017

(51) Int. Cl. A61K 31/202; A61P 7/00

(52) U.S. Cl. CPC A61K 31/202; A61P 7/00

(57) ABSTRACT

Methods are disclosed to reduce apolipoprotein C-III (apoC-III) mRNA or protein in a subject in need thereof, comprising administering a pharmaceutically effective amount of a compound of Formula (I), or a pharmaceutically acceptable salt or ester thereof, wherein R₁ and R₂ are independently chosen from a hydrogen atom or linear, branched, and/or cyclic C₁-C₆ alkyl groups, with the proviso that R₁ and R₂ are not both hydrogen.

The present disclosure relates to a method of reducing apolipoprotein C-III (apoC-III) mRNA or protein in a subject in need thereof, comprising administering to the subject a pharmaceutically effective amount of a compound of Formula (I):

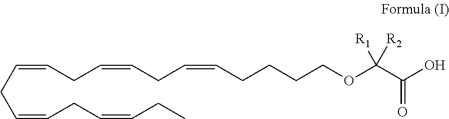

or a pharmaceutically acceptable salt or ester thereof,

wherein R₁ and R₂ are independently chosen from a hydrogen atom or linear, branched, and/or cyclic C₁-C₆ alkyl groups, with the proviso that Rand R₂ are not both hydrogen. Such methods, compounds, and compositions are useful to treat conditions caused by, associated with, or aggravated by, elevated hepatic and/or plasma apoC-III such as hypertriglyceridemia (HTG), hyperchylomicronemia, dyslipidemia, pancreatitis and in the prevention and/or treatment of one or more of cardiovascular disease or metabolic disorder, or a symptom thereof.

Dietary polyunsaturated fatty acids (PUFAs), including omega-3 fatty acids, have effects on diverse physiological processes impacting normal health and chronic diseases, such as the regulation of plasma lipid levels, cardiovascular and immune functions, insulin action, neuronal development, and visual function.

Omega-3 fatty acids, e.g., (5Z,8Z,11Z,14Z,17Z)-icosa-5,8,11,14,17-pentaenoic acid (EPA) and (4Z,7Z,10Z,13Z,16Z,19Z)-docosa-4,7,10,13,16,19-hexaenoic acid (DHA), regulate plasma lipid levels, cardiovascular and immune functions, insulin action, and neuronal development, and visual function. Omega-3 fatty acids have been shown to have beneficial effects on the risk factors for cardiovascular diseases, for example hypertension and hypertriglyceridemia (HTG), and on the coagulation factor VII phospholipid complex activity.

WO 2010/128401 discloses that 2-((5Z,8Z,11Z,14Z,17Z)-icosa-5,8,11,14,17-pentaenyloxy)butanoic acid favorably influences lipid profiles and inhibits i.a. development of atherosclerosis, decreases total cholesterol and increases HDL cholesterol as compared to a control. Those results demonstrate that 2-((5Z,8Z,11Z,14Z,17Z)-icosa-5,8,11,14,17-pentaenyloxy)butanoic acid and its derivatives may be useful in the prevention or treatment of various conditions, such as inflammation, hyperlipidemic conditions, obesity, fatty liver disease, atherosclerosis, peripheral insulin resistance, and/or diabetic conditions. Further use of 2-((5Z,8Z,11Z,14Z,17Z)-icosa-5,8,11,14,17-pentaenyloxy)butanoic acid and its derivatives for treating different diseases or conditions is disclosed in WO 2012/059818.

More particularly WO2012/059818 describes a method of treating or preventing at least one disease or condition selected from elevated Apo B, primary hypercholesterolemia (heterozygous familial and nonfamilial), and primary dysbetalipoproteinemia (Fredrickson Type III) in a subject in need thereof, comprising administering to the subject a pharmaceutically effective amount of a compound of Formula (I). However although it is already established that Apo B and Apo E (dysbetalipoproteinemia) related pathways are positively affected by compounds of Formula (I), data from 2 clinical studies in distinct patient populations surprisingly revealed that an additional apolipoprotein, apoC-III, is also potently reduced by compounds of Formula (I).

ApoC-III is a glycoprotein produced primarily by the liver whose function is believed to involve promoting the assembly and secretion of triglyceride-rich VLDL particles from hepatic cells under lipid-rich conditions (Sundaram M et al., J Lipid Research, vol. 51, 2010). In plasma it is largely associated with very low-density lipoprotein (VLDL), high-density lipoprotein (HDL) and chylomicrons. An increase in apoC-III levels induces the development of hypertriglyceridemia. The mechanisms by which apoC-III expression increase plasma triglycerides are partially mediated via inhibition of lipoprotein lipase and hepatic lipase; it thereby delays the catabolism of triglyceride-rich particles. ApoC-III is also thought to inhibit hepatic uptake of triglyceride rich particles. The clinical importance of apoC-III has been established by studies demonstrating that carriers of rare mutations that disrupt apoC-III function have both lower TG levels and a reduced risk of coronary/ischemic heart disease (N Engl j Med. 2014 Jul 3; 371(1):22-31, Loss-of-function mutations in APOC3, triglycerides, and coronary disease).

The long-chain omega-3 fatty acids, EPA and DHA, are well established in the treatment of HTG. Given the recent identification of apoC-III as both a pivotal regulator in triglyceride levels and as a genetically validated target for the prevention of coronary heart disease, the effects of omega-3 fatty acids in various forms and compositions upon plasma apoC-III levels have been investigated. By way of example US2014/0221486 claims a method for reducing an apoC-III level of a subject either on statin therapy and having baseline fasting triglycerides of about 200 mg/dl to about 499 mg/dl, or a subject having fasting baseline triglycerides of at least about 500 mg/dl, by administering a pharmaceutical composition comprising about 1 g to about 4 g of ethyl eicosapentaenoate per day to the subject. US 2013/0177643 claims a method of lowering serum or plasma apoC-III levels, comprising administering a pharmaceutical composition comprising: EPA, substantially in free acid form, in an amount of at least about 50% (a/a); DHA, substantially in free acid form, in an amount of at least about 15% (a/a); DPA, substantially in free acid form, in an amount of at least about 1% (a/a); in an amount and for a duration sufficient to reduce serum or plasma apoC-III from pre-treatment levels. Yet another example can be found in US2014/0094520 claiming a method of reducing a lipid parameter level in a subject from a baseline lipid parameter level, wherein the lipid parameter is selected from a group consisting of inter alia apoC-III, comprising administering to the subject a composition comprising fatty acids, wherein at least 50 percent by weight of the fatty acids comprise omega-3 fatty acids, salts, esters, or derivatives thereof, wherein the omega-3 fatty acids comprise eicosapentaenoic acid (EPA), docosapentaenoic acid (DPA) and wherein the ratio of docosahexaenoic acid to DHA to EPA (DHA:EPA) is less than 1:10, and wherein the ratio of DHA to DPA (DHA:DPA) is less than 2:1.

Effective lowering of hepatic/plasma apoC-III with an orally delivered omega-3/omega-3 derivative offers an attractive treatment option for selected patient populations if clinically relevant reductions can be achieved. Although it is yet to be determined what degree of reduction in apoC-III is ‘clinically relevant’, studies in subjects with loss-of-function apoC-III mutations show that apoC-III levels 46% lower than non-carriers are associated with a 40% lower risk of coronary heart disease (CHD) (N Engl J Med. 2014 Jul 3; 371(1):22-31, Loss-of-function mutations in APOC3, triglycerides, and coronary disease). In addition to the reduced apoC-III concentrations, carriers also had 39% lower TG concentrations than non-carriers. Given that loss-of-function represents life-long exposure, it is therefore conceivable that therapies aimed at reducing apoC-III over a shorter time frame should aim for apoC-III reductions as close to (or higher) than those associated with loss-of-function mutations if beneficial effects upon CHD are to be achieved. As the apoC-III results achieved with naturally occurring omega-3 lipids are relatively modest (see Example 26), compounds that more potently reduce apoC-III may offer not only superior triglyceride lowering but also superior cardioprotective effects.

The present disclosure relates to a method of reducing apolipoprotein C-III (apoC-III) mRNA or protein in a subject in need thereof, comprising administering to the subject a pharmaceutically effective amount of a compound of Formula (I):

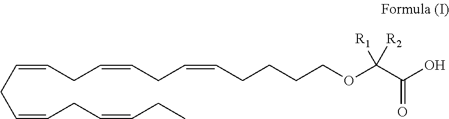

or a pharmaceutically acceptable salt or ester thereof,
wherein R₁ and R₂ are independently chosen from a hydrogen atom or linear, branched, and/or cyclic C₁-C₂ alkyl groups, with the proviso that R₁ and R₂ are not both hydrogen.

A number of metabolic diseases or conditions are closely associated with increased risk of cardiovascular events. Such diseases or conditions include, but are not limited to, diabetes mellitus type I and type II, metabolic syndrome, dyslipidemic conditions such as hypercholesterolemia, hyperlipidemia, mixed dyslipidemia, hypertriglyceridemia, hyperchyolomicronemia, and various familial dyslipidemias.

In at least one embodiment the disease or condition is chosen from any of hypertriglyceridemia (HTG), hyperchylomicronemia, dyslipidemia, and pancreatitis and in the prevention and/or treatment of one or more of cardiovascular disease or metabolic disorder, or a symptom thereof.

The present disclosure also includes a method of reducing apoC-III in a subject in need thereof, the method comprising administering to the subject a pharmaceutically effective amount of 2-((5Z,8Z,11Z,14Z,17Z)-icosa-5,8,11,14,17-pentaen-1-yloxy)butanoic acid:

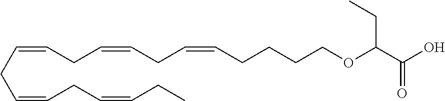 [SMILES: CC/C=C\C/C=C\C/C=C\C/C=C\C/C=C\CCCCOC(CC)C(=O)O]

or a pharmaceutically acceptable salt or ester thereof.

### BRIEF DESCRIPTION OF THE DRAWINGS

FIG. 1 discloses the relative hepatic apoC-III gene expression for a compound of Formula (I), a control, and a reference compound.

### DESCRIPTION

Particular aspects of the disclosure are described in greater detail below. The terms and definitions as used in the present application and as clarified herein are intended to represent the meaning within the present disclosure.

The singular forms “a,” “an,” and “the” include plural reference unless the context dictates otherwise.

The terms “approximately” and “about” mean to be nearly the same as a referenced number or value. As used herein, the terms “approximately” and “about” should be generally understood to encompass ±5% of a specified amount, frequency, or value.

The terms “treat,” “treating,” and “treatment” include any therapeutic application that can benefit a human or non-human mammal. Both human and veterinary treatments are within the scope of the present disclosure. Treatment may be responsive to an existing condition or it may be prophylactic, i.e., preventative.

The terms “administer,” “administration,” and “administering” as used herein refer to (1) providing, giving, dosing and/or prescribing by either a health practitioner or his authorized agent or under his direction a compound or composition according to the present disclosure, and (2) putting into, taking or consuming by the human patient or person himself or herself, or non-human mammal a compound or composition according to the present disclosure.

The term “pharmaceutically effective amount” means an amount sufficient to achieve the desired pharmacological and/or therapeutic effects, i.e., an amount of the disclosed compound that is effective for its intended purpose. While individual subject/patient needs may vary, the determination of optimal ranges for effective amounts of the disclosed compound is within the skill of the art. Generally, the dosage regimen for treating a disease and/or condition with the compounds presently disclosed may be determined according to a variety of factors such as the type, age, weight, sex, diet, and/or medical condition of the subject/patient.

The term “pharmaceutical composition” means a compound according to the present disclosure in any form suitable for medical use.

The compounds of Formula (I) may exist in various stereoisomeric forms, including enantiomers, diastereomers, or mixtures thereof. It will be understood that the invention encompasses all optical isomers of the compounds of Formula (I) and mixtures thereof. Hence, compounds of Formula (I) that exist as diastereomers, racemates, and/or enantiomers are within the scope of the present disclosure.

The present disclosure relates to a method of reducing apolipoprotein C-III (apoC-III) mRNA or protein in a subject in need thereof, comprising administering to the subject a pharmaceutically effective amount of a compound of Formula (I):

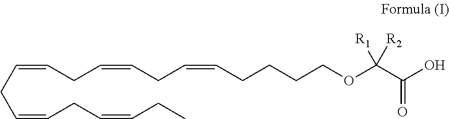

or a pharmaceutically acceptable salt or ester thereof,
wherein R₁ and R₂ are independently chosen from a hydrogen atom or linear, branched, and/or cyclic C₁-C₆ alkyl groups, with the proviso that R₁ and R₂ are not both hydrogen.

In at least one embodiment, the present disclosure relates to use of a pharmaceutically effective amount of a compound of Formula (I):

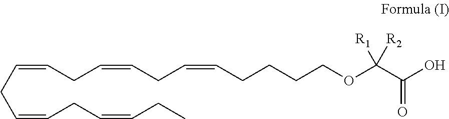

or a pharmaceutically acceptable salt or ester thereof, for reducing apolipoprotein C-III

(apoC-III) mRNA or protein in a subject in need thereof,

wherein R₁ and R₂ are independently chosen from a hydrogen atom or linear, branched, and/or cyclic C₁-C₆ alkyl groups, with the proviso that R₁ and R₂ are not both hydrogen.

In at least one embodiment, R₁ and R₂ are chosen from a hydrogen atom, a methyl group, an ethyl group, a n-propyl group, and an isopropyl group.

In at least one embodiment, R₁ and R₂ are chosen from a hydrogen atom, a methyl group, and an ethyl group.

In at least one embodiment, one of R₁ and R₂ is a hydrogen atom and the other one of R₁ and R₂ is chosen from a C₁-C₃ alkyl group. In one embodiment one of R₁ and R₂ is a hydrogen atom and the other one of R₁ and R₂ is chosen from a methyl group or an ethyl group.

In at least one embodiment, the compound is present in its various stereoisomeric forms, such as an enantiomer (R or S), diastereomer, or mixtures thereof.

In at least one embodiment, the compound is present in racemic form.

In cases, where the compound according to Formula (I) is a salt of a counter-ion with at least one stereogenic center, or ester of an alcohol with at least one stereogenic center, the compound may have multiple stereocenters. In those situations, the compounds of the present disclosure may exist as diastereomers. Thus, in at least one embodiment, the compounds of the present disclosure are present as at least one diastereomer.

In at least one embodiment, the compound of the present disclosure is 2-((5Z,8Z,11Z, 14Z,17Z)-icosa-5,8,11,14,17-pentaen-1-yloxy)butanoic acid:

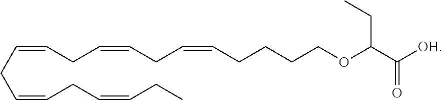 [SMILES: CC/C=C\C/C=C\C/C=C\C/C=C\C/C=C\CCCCOC(CC)C(=O)O]

In at least one embodiments the compound of the present disclosure is present in its S and/or R form represented by the formulas:

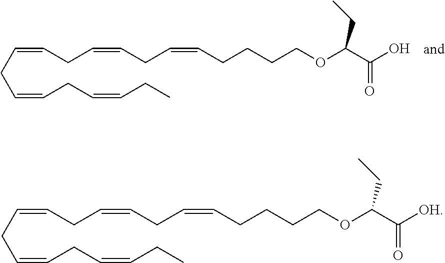 [SMILES: CC/C=C\C/C=C\C/C=C\C/C=C\C/C=C\CCCCO[C@@H](CC)C(=O)O ; CC/C=C\C/C=C\C/C=C\C/C=C\C/C=C\CCCCO[C@H](CC)C(=O)O]

In at least one embodiment the disease or condition is chosen from any of hypertriglyceridemia (HTG), hyperchylomicronemia, dyslipidemia, and pancreatitis and in the prevention and/or treatment of one or more of cardiovascular disease or metabolic disorder, or a symptom thereof. In one embodiment the disease or condition is chosen from any of hyperchylomicronemia, pancreatitis and in the prevention and/or treatment of one or more of cardiovascular disease or metabolic disorder, or a symptom thereof. In one embodiment the disease or condition is chosen from any of hyperchylomicronemia and pancreatitis.

Compounds of Formula (I) can be prepared as described, for example, in PCT Application WO 2010/128401 filed May 7, 2010, and according to Examples 1-23 below.

Examples 1-23 are exemplary and one skilled in the art would understand how to apply these general methods to arrive at other compounds within the scope of Formula (I). Compounds of the present disclosure may be in the form of a pharmaceutically acceptable salt or ester. For example, the compounds of Formula (I) may be in the form of esters, such as a phospholipid, a glyceride or a C₁-C₆-alkyl ester. In at least one embodiment, the ester is chosen from a glyceride or a C₁-C₆-alkyl ester. In at least one embodiment, the ester is chosen from a triglyceride, a 1,2 diglyceride, a 1,3-diglyceride, a 1-monoglyceride, a 2-monoglyceride, a methyl ester, an ethyl ester, a propyl ester, a isopropyl ester, a n-butyl ester and a tert-butyl ester. In at least one embodiment, the compound of Formula (I) is present as a methyl ester, an ethyl ester, an isopropyl ester, a n-butyl ester or a tert-butyl ester, for example as a methyl ester or an ethyl ester. It has been proven by in-vitro digestion studies in a bio relevant media that esters represented by Formula (I) (i.e., the ethyl ester and the butyl ester) will be rapidly hydrolyzed in the gastrointestinal tract.

Salts suitable for the present disclosure include, but are not limited to, salts of NH⁴⁺; metal ions such as Li⁺, Na⁺Mg²⁺, or Ca²⁺; a protonated primary amine such as tert-butyl ammonium, (3S,5S,7S)-adamantan-1-ammonium, 1,3-dihydroxy-2-(hydroxymethyl)propane-2-ammonium, a protonated aminopyridine (e.g., pyridine-2-ammonium); a protonated secondary amine such as diethylammonium, 2,3,4,5,6-pentahydroxy-N-methylhexan-1-ammonium, N-ethylnaphthalen-1-ammonium, a protonated tertiary amine such as 4-methylmorpholin-4-ium, a protonated quaternary amine such as 2-hydroxy-N,N,N-trimethylethan-1-ammonium and a protonated guanidine such as amino((4-amino-4-carboxybutyl)amino)methaniminium or a protonated heterocycle such as 1H-imidazol-3-ium. Additional examples of suitable salts include salts of a diprotonated diamine such as ethane-1,2-diammonium or piperazine-1,4-diium. Other salts according to the present disclosure may comprise protonated Chitosan:

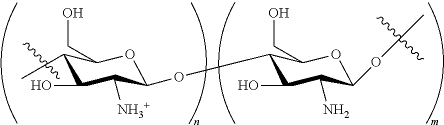

In at least embodiment the salts are chosen from a sodium salt, a calcium salt, and a choline salt.

The present disclosure provides for a method of reducing apoC-III in a subject in need thereof, comprising administering to the subject a pharmaceutically effective amount of a compound of Formula (I). The subject may be a human or a non-human mammal. The compounds presently disclosed may be administered as a medicament, such as in a pharmaceutical composition.

In at least one embodiment, the present disclosure relates to a method for reducing an apoC-III level of a subject on statin therapy and having baseline fasting triglycerides of about 200 mg/dl to about 499 mg/dl by administering to the subject a pharmaceutical effective amount of a compound of Formula (I). In another embodiment the present disclosure relates to use of a pharmaceutical effective amount of a compound of Formula (I), in the manufacture of a medicament for reducing an apoC-III level of a subject on statin therapy and having baseline fasting triglycerides of about 200 mg/dl to about 499 mg/dl. The apoC-III level can be reduced by at least about 20%, by at least about 25%, by at least about 30% or by at least about 35%.

In at least one embodiment, the disclosure relates to a method for reducing an apoC-III level of a subject having baseline fasting triglycerides of about 200 mg/dl to about 499 mg/dl by administering to the subject a pharmaceutical effective amount of a compound of Formula (I). In another embodiment the present disclosure relates to use of a pharmaceutical effective amount of a compound of Formula (I), in the manufacture of a medicament for reducing an apoC-III level of a subject having baseline fasting triglycerides of about 200 mg/dl to about 499 mg/dl. The apoC-III level can be reduced by at least about 20%, by at least about 25%, by at least about 30% or by at least about 35%.

In at least one embodiment, the present disclosure relates to a method for reducing an apoC-III level of a subject on statin therapy and having baseline fasting triglycerides of above 500 mg/dl by administering to the subject a pharmaceutical effective amount of a compound of Formula (I). In another embodiment the present disclosure relates to use of a pharmaceutical effective amount of a compound of Formula (I), in the manufacture of a medicament for reducing an apoC-III level of a subject on statin therapy and having baseline fasting triglycerides of above 500 mg/dl. The apoC-III level can be reduced by at least about 25%, by at least about 30%, by at least about 35% or by at least about 40%.

In at least one embodiment, the disclosure relates to a method for reducing an apoC-III level of a subject having baseline fasting triglycerides of above 500 mg/dl by administering to the subject a pharmaceutical effective amount of a compound of Formula (I). In another embodiment the present disclosure relates to use of a pharmaceutical effective amount of a compound of Formula (I), in the manufacture of a medicament for reducing an apoC-III level of a subject having baseline fasting triglycerides of above 500 mg/dl. The apoC-III level can be reduced by at least about 25%, by at least about 30%, by at least about 35% or by at least about 40%.

The present disclosure also relates to a method for reducing an apoC-III level of a subject having baseline fasting LDL-cholesterol of at least 2.5 mmol/L (˜97 mg/dl) by administering to the subject a pharmaceutical effective amount of a compound of Formula (I). In another embodiment the present disclosure relates to use of a pharmaceutical effective amount of a compound of Formula (I), in the manufacture of a medicament for reducing an apoC-III level of a subject having baseline fasting LDL-cholesterol of at least 2.5 mmol/L (˜97 mg/dl). The apoC-III level can be reduced by at least about 25%, by at least about 30%, by at least about 35% or by at least about 40%.

In at least one embodiment the present disclosure relates to a method for reducing apoC-III in a subject in need thereof, comprising administering to the subject a pharmaceutically effective amount of a dyslipidemic agent such as for example a statin and a compound of Formula (I).

The composition presently disclosed may comprise at least one compound of Formula (I) and optionally at least one non-active pharmaceutical ingredient, i.e., excipient. Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. Examples of excipients include, but are not limited to, solvents, carriers, diluents, binders, fillers, sweeteners, aromas, pH modifiers, viscosity modifiers, antioxidants, extenders, humectants, disintegrating agents, solution-retarding agents, absorption accelerators, wetting agents, absorbents, lubricants, coloring agents, dispersing agents, and preservatives. Excipients may have more than one role or function, or may be classified in more than one group; classifications are descriptive only and are not intended to be limiting. In some embodiments, for example, the at least one excipient may be chosen from corn starch, lactose, glucose, microcrystalline, cellulose, magnesium stearate, polyvinylpyrrolidone, citric acid, tartaric acid, water, ethanol, glycerol, sorbitol, polyethylene glycol, propylene glycol, cetylstearyl alcohol, carboxymethylcellulose, and fatty substances such as hard fat or suitable mixtures thereof. In some embodiments, the compositions presently disclosed comprise at least one compound of Formula (I) and at least one pharmaceutically acceptable antioxidant, e.g., tocopherol such as alpha-tocopherol, beta-tocopherol, gamma-tocopherol, and delta-tocopherol, or mixtures thereof, BHA such as 2-tert-butyl-4-hydroxyanisole and 3-tert-butyl-4-hydroxyanisole, or mixtures thereof and BHT (3,5-di-tert-butyl-4-hydroxytoluene), or mixtures thereof.

The compositions presently disclosed may be formulated in oral administration forms, e.g., tablets or gelatin soft or hard capsules. The dosage form can be of any shape suitable for oral administration, such as spherical, oval, ellipsoidal, cube-shaped, regular, and/or irregular shaped. Conventional formulation techniques known in the art, may be used to formulate the compounds according to the present disclosure. In some embodiments, the composition may be in the form of a gelatin capsule or a tablet.

A suitable daily dosage of a compound of Formula (I) may range from about 5 mg to about 2 g. For example, in some embodiments, the daily dose ranges from about 50 mg to about 1 g, from about 100 mg to about 1 g, from about 50 mg to about 800 mg, from about 100 mg to about 800 mg, from about 100 mg to about 600 mg. In at least one embodiment, the daily dose ranges from about 200 mg to about 600 mg. The compounds may be administered, for example, once, twice, or three times per day. In at least one embodiment, the compound of Formula (I) is administered in an amount ranging from about 200 mg to about 800 mg per dose. In at least one embodiment, the compound of Formula (I) is administered once per day.

The present inventors have found that compounds of Formula (I), such as 2-((5Z,8Z,11Z,14Z,17Z)-icosa-5,8,11,14,17-pentaen-1-yloxy)butanoic acid, have remarkably good pharmaceutical activity. Surprisingly, the compounds of Formula (I) presently disclosed exhibit improved biological activity compared to naturally occurring omega-3 fatty acids, such as EPA and DHA for reducing apoC-III.

In some embodiments, for example, compounds of Formula (I) may reduce the median levels of apoC-III in plasma or in the liver by at least 25-30% versus baseline, i.e., a superior decrease to that achieved with available EPA/DHA/DPA combinations. As compounds of Formula (I) have been shown to decrease hepatic apoC-III mRNA in pre-clinical models (and thus presumably also hepatic production/secretion), the addition of lipid-lowering drugs that reduce apoC-III via increased hepatic uptake of apo B particles, e.g., statins or PCSK-9 inhibitors, could be expected to exert additional plasma apoC-III lowering effects.

### EXAMPLES

The present disclosure may be further described by the following non-limiting examples, in which standard techniques known to the skilled chemist and techniques analogous to those described in these examples may be used where appropriate. It is understood that the skilled artisan will envision additional embodiments consistent with the disclosure provided herein.

Unless otherwise stated, reactions were carried out at room temperature, typically in the range between 18-25° C. with solvents of HPLC grade under anhydrous conditions. Evaporations were carried out by rotary evaporation in vacuo. Column chromatography was performed by the flash procedure on silica gel. Nuclear magnetic resonance (NMR) shift values were recorded on a Bruker Avarice DPX 200 or 300, or on an AVII 400 instrument with peak multiplicities described as follows: s, singlet; d, doublet; dd, double doublet; t, triplet; q, quartet; p, pentet; m, multiplett; br, broad. Mass spectra were recorded with a G1956A mass spectrometer (electrospray, 3000 V) switching positive and negative ionization mode. Reported yields are illustrative and do not necessarily represent the maximum yield attainable.

### Example 1: Preparation of tert-butyl 2-((5Z,8Z,11Z,14Z,17Z)-icosa- 5,8,11,14,17-pentaen-1-yloxy)butanoate

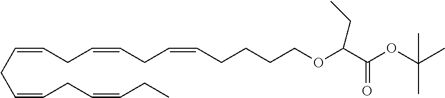 [SMILES: CC/C=C\C/C=C\C/C=C\C/C=C\C/C=C\CCCCOC(CC)C(=O)OC(C)(C)C]

Tetrabutylammonium chloride (0.55 g, 1.98 mmol) was added to a solution of (5Z,8Z,11Z,14Z,17Z)-icosa-5,8,11,14,17-pentaen-1-ol, (3.50 g, 12.1 mmol) in toluene (35 mL) at room temperature under nitrogen. An aqueous solution of sodium hydroxide (50% (w/w), 11.7 mL) was added under vigorous stirring at room temperature, followed by t-butyl 2-bromobutyrate (5.41 g, 24.3 mmol). The resulting mixture was heated to 50° C and additional t-butyl 2-bromobutyrate was added after 1.5 hours (2.70 g, 12.1 mmol), 3.5 hours (2.70 g, 12.1 mmol) and 4.5 hours (2.70 g, 12.1 mmol) and stirred for 12 hours in total. After cooling to room temperature, ice water (25 mL) was added and the resulting two phases were separated. The organic phase was washed with a mixture of NaOH (5%) and brine, dried (MgSO₄), filtered and concentrated. The residue was purified by flash chromatography on silica gel using increasingly polar mixtures of heptane and ethyl acetate (100:0->95:5) as eluent. Concentration of the appropriate fractions afforded 1.87 g (36% yield) of the title compound as an oil. ¹H NMR (300 MHz, CDCI 3): δ 0.85-1.10 (m, 6H), 1.35-1.54 (m, 11H), 1.53-1.87 (m, 4H), 1.96-2.26 (m, 4H), 2.70-3.02 (m, 8H), 3.31 (dt, 1H), 3.51-3.67 (m, 2H), 5.10-5.58 (m, 10H).

### Example 2: Preparation of tert-butyl 2-((5Z,8Z,11Z,14Z,17Z)-icosa-5,8,11,14,17-pentaenyloxy)butanoate acid (Compound A)

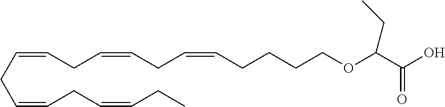 [SMILES: CC/C=C\C/C=C\C/C=C\C/C=C\C/C=C\CCCCOC(CC)C(=O)O]

tert-Butyl 2-((5Z,8Z,11Z,14Z,17Z)-icosa-5,8,11,14,17-pentaen-1-yloxy)butanoate (19.6 g, 45.5 mmol) was dissolved in dichloromethane (200 mL) and placed under nitrogen. Trifluoroacetic acid (50 mL) was added and the reaction mixture was stirred at room temperature for one hour. Water was added and the aqueous phase was extracted twice with dichloromethane. The combined organic extract was washed with brine, dried (Na₂SO₄), filtered and concentrated. The residue was subjected to flash chromatography on silica gel using increasingly polar mixtures of heptane, ethyl acetate and formic acid (90: 10:1->80:20:1) as eluent. Concentration of the appropriate fractions afforded 12.1 g (71% yield) of the title compound as an oil. ¹H-NMR (300 MHz, CDCl₃): δ 0.90-1.00 (m, 6H), 1.50 (m, 2H), 1.70 (m, 2H), 1.80 (m, 2H), 2.10 (m, 4H), 2.80-2.90 (m, 8H), 3.50 (m, 1H), 3.60 (m, 1H), 3.75 (t, 1H), 5.30-5.50 (m, 10H); MS (electrospray): 373.2 [M-H]⁻.

### Example 3: Preparation of (4S,5R)-3-((S)-2-((5Z,8Z11Z,14Z,17Z)-icosa-5,8,11,14,17-pentaenyloxy)butanoyl)-4-methyl-5-phenyloxazolidin-2-one and (4S,5R)-3-((R)-2-((5Z,8Z,11Z,14Z,17Z)-icosa-5,8,11,14,17-pentaenyloxy)butanoyl)-4-methyl-5-phenyloxazolidin-2-one

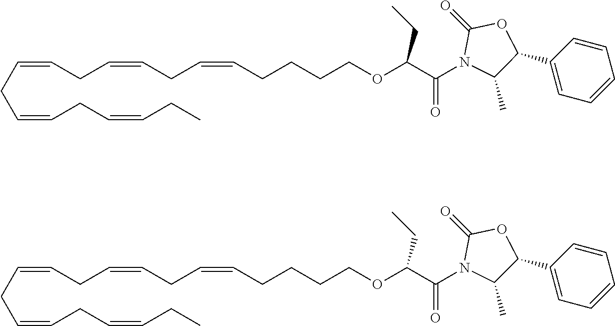 [SMILES: CC/C=C\C/C=C\C/C=C\C/C=C\C/C=C\CCCCO[C@@H](CC)C(=O)N1C(=O)O[C@H](c2ccccc2)[C@@H]1C ; CC/C=C\C/C=C\C/C=C\C/C=C\C/C=C\CCCCO[C@H](CC)C(=O)N1C(=O)O[C@H](c2ccccc2)[C@@H]1C]

DMAP (1.10 g, 8.90 mmol) and DCC (1.90 g, 9.30 mmol) were added to a mixture of 2-((5Z,8Z,11Z,14Z,17Z)-icosa-5,8,11,14,17-pentaenyloxy)butanoic acid (3.20 g, 8.50 mmol) in dry dichloromethane (100 mL) held at 0° C. under nitrogen. The resulting mixture was stirred at 0° C. for 20 minutes. (4S,5R)-4-methyl-5-phenyloxazolidin-2-one (1.50 g, 8.50 mmol) was added and the resulting turbid mixture was stirred at ambient temperature for five days. The mixture was filtrated and concentrated under reduced pressure to give a crude product containing the desired product as a mixture of two diastereomers. The residue was purified by flash chromatography on silica gel using 15% ethyl acetate in heptane as eluent. The two diastereomers were separated and the appropriate fractions were concentrated. (4S,5R)-3-((S)-2-((5Z,8Z,11Z,14Z,17Z)-icosa-5,8,11,14,17-pentaenyloxy)butanoyl)-4-methyl-5-phenyloxazolidin-2-one eluted first and was obtained in 1.1 g (40% yield) as an oil. (4S,5R)-3-((R)-2-((5Z,8Z,11Z,14Z,17Z)-icosa-5,8,11,14,17-pentaenyloxy)butanoyl)-4-methyl-5-phenyloxazolidin-2-one was obtained in 0.95 g (34% yield) as an oil.

(4S,5R)-3-((S)-2-((5Z,8Z,11Z,14Z,17Z)-icosa-5,8,11,14,17-pentaenyloxy)butanoyl)-4-methyl-5-phenyloxazolidin-2-one (E1): ¹H-NMR (300 MHz, CDCl₃): δ 0.90 (d, 3H), 1.00 (t, 3H), 1.07 (t, 3H), 1.45-1.57 (m, 2H), 1.62-1.76 (m, 3H), 1.85-1.95 (m, 1H), 2.05-2.15 (m, 4H), 2.87 (m, 8H), 3.39 (m, 1H), 3.57 (m, 1H), 4.85-4.92 (m, 2H), 5.30-5.45 (m, 10H), 5.75 (d, 1H), 7.32 (m, 2H), 7.43 (m, 3H).

(4S,5R)-3-((R)-2-((5Z,8Z,11Z,14Z,17Z)-icosa-5,8,11,14,17-pentaenyloxy)butanoyl)-4-methyl-5-phenyloxazolidin-2-one (E2) ¹H-NMR (300 MHz, CDCl₃): δ 0.98 (d, 3H), 0.99 (t, 3H), 1.08 (t, 3H), 1.40-4.52 (m, 2H), 1.55-1.75 (m, 3H), 1.80-1.90 (m, 1H), 2.05-2.15 (m, 4H), 2.84 (m, 8H), 3.39 (m, 1H), 3.56 (m, 1H), 4.79 (pent, 1H), 4.97 (dd, 1H) 5.30-5.45 (m, 10H), 5.71 (d, 1H), 7.33 (m, 2H), 7.43 (m, 3H).

### Example 4: Preparation of (S)-2-((5Z,8Z,11Z,14Z,17Z)-icosa-5,8,11,14,17-pentaenyloxy)butanoic acid

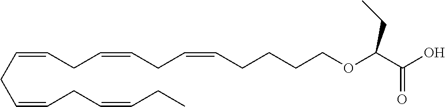 [SMILES: CC/C=C\C/C=C\C/C=C\C/C=C\C/C=C\CCCCO[C@@H](CC)C(=O)O]

Hydrogen peroxide (35% in water, 0.75 mL, 8.54 mmol) and lithium hydroxide monohydrate (0.18 g, 4.27 mmol) was added to a solution of (4S,5R)-3-((S)-2-((5Z,8Z,11Z,14Z,17Z)-icosa-5,8,11,14,17-pentaenyloxy)butanoyl)-4-methyl-5-phenyloxazolidin-2-one (1.10 g, 2.13 mmol) in tetrahydrofuran (12 mL) and water (4 mL) held at 0° C. under nitrogen. The reaction mixture was stirred at 0° C. for 30 minutes. 10% Na2SO3 (aq) (30 mL) was added, the pH was adjusted to ˜2 with 2M HCl and the mixture was extracted twice with heptane (30 mL). The combined organic extract was dried (Na₂SO₄), filtered and concentrated. The residue was subjected to flash chromatography on silica gel using increasingly polar mixtures of heptane and ethyl acetate (98:8->1:1) as eluent. Concentration of the appropriate fractions afforded 0.48 g (60% yield) of the title compound as an oil. ¹H-NMR (300 MHz, CDCl₃): δ 0.90-1.00 (m, 6H), 1.48 (m, 2H), 1.65 (m, 2H), 1.85 (m, 2H), 2.10(m, 4H), 2.80-2.90 (m, 8H), 3.55 (m, 1H), 3.60 (m, 1H), 3.88 (t, 1H), 5.35-5.45 (m, 10H); MS (electrospray): 373.3 [M-H]⁻; [?]_{D} −31° (c=0.088, ethanol).

### Example 5: Preparation of (R)-2-((5Z,8Z,11Z,14Z,17Z)-icosa-5,8,11,14,17-pentaenyloxy)butanoic acid

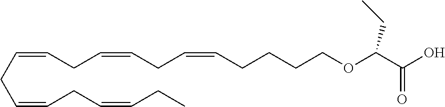 [SMILES: CC/C=C\C/C=C\C/C=C\C/C=C\C/C=C\CCCCO[C@H](CC)C(=O)O]

Hydrogen peroxide (35% in water, 0.65 mL, 7.37 mmol) and lithium hydroxide monohydrate (0.15 g, 3.69 mmol) was added to a solution of (4S,5R)-3-((R)-2-((5Z,8Z,11Z,14Z,17Z)-icosa-5,8,11,14,17-pentaenyloxy)butanoyl)-4-methyl-5-phenyloxazolidin-2-one (0.95 g, 1.84 mmol) in tetrahydrofuran (12 mL) and water (4 mL) held at 0° C. under nitrogen. The reaction mixture was stirred at 0° C. for 30 minutes. 10% Na₂SO₃ (aq) (30 mL) was added, the pH was adjusted to 2 with 2M HCl and the mixture was extracted twice with heptane (30 mL). The combined organic extract was dried (Na₂SO₄), filtered and concentrated. The residue was subjected to flash chromatography on silica gel using increasingly polar mixtures of heptane and ethyl acetate (98:8->50:50) as eluent. Concentration of the appropriate fractions afforded 0.19 g (29% yield) of the title compound as an oil. 1H-NMR (300 MHz, CDCl₃): δ 0.90-1.00 (m, 6H), 1.48 (m, 2H), 1.65 (m, 2H), 1.85 (m, 2H), 2.10 (m, 4H), 2.80-2.90 (m, 8H), 3.55 (m, 1H), 3.60 (m, 1H), 3.88 (t, 1H), 5.35-5.45 (m, 10H); MS (electrospray): 373.3 [M-H]⁻; [?]_{D} −31° (c=0.088, ethanol).

### Example 6: Preparation of tert-butyl 2-((5Z,8Z,11.Z,14Z,17Z)-icosa-5,8,11,14,17-pentaenyloxy)propanoate

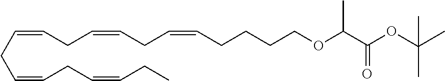 [SMILES: CC/C=C\C/C=C\C/C=C\C/C=C\C/C=C\CCCCOC(C)C(=O)OC(C)(C)C]

A mixture of (5Z,8Z,11Z,14Z,17Z)-icosa-5,8,11,14,17-pentaen-1-ol, (1.00 g, 3.47 mmol), tetrabutylammonium chloride (0.24 g, 0.87 mmol) and t-butyl 2-bromopropionate (3.62 g, 17.3 mmol) was dissolved in toluene (36 mL) and placed under nitrogen. An aqueous solution of sodium hydroxide (50%, 8 mL) was added slowly under vigorous stirring and the resulting mixture was stirred at ambient temperature for twenty hours. Water was added and the mixture was extracted three times with ether. The combined organic extract was washed with brine, dried (Na₂SO₄), filtered and concentrated. The residue was purified by flash chromatography on silica gel using 2% ethyl acetate in heptane as eluent. Concentration of the appropriate fractions afforded 1.40 g (90% yield) of the title compound as an oil. ¹H-NMR (300 MHz, CDCl ₃): δ 0.95 (t, 3H), 1.41 (d, 3H), 1.48 (s, 9H), 1.48-1.66 (m, 4H), 2.05 (m, 4H), 2.83 (m, 8H), 3.35 (m, 1H), 3.55 (m, 1H), 3.79 (q, 1H), 5.32-5.44 (m, 10H).

### Example 7: Preparation of 2-((5Z,8Z,11Z,14Z,17Z)-icosa-5,8,11,14,17-pentaenyloxy)propanoic acid

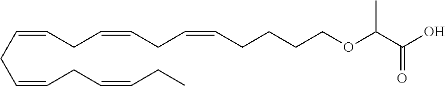 [SMILES: CC/C=C\C/C=C\C/C=C\C/C=C\C/C=C\CCCCOC(C)C(=O)O]

Trifluoroacetic acid (2 mL) was added to a solution of 2-((5Z,8Z,11Z,14Z,17Z)-icosa-5,8,11,14,17-pentaenyloxy)propanoate (1.40 g, 3.36 mmol) in dichloromethane (10 mL) held under nitrogen and the reaction mixture was stirred at room temperature for three hours. Diethyl ether (50 mL) was added and the organic phase was washed with water (30 mL), dried (Na₂SO₄) and concentrated. The residue was subjected to flash chromatography on silica gel using increasingly polar mixtures of heptane, ethyl acetate and formic acid (95:5:0.25->80:20:1) as eluent. Concentration of the appropriate fractions afforded 0.67 g of slightly impure product. This material was dissolved in heptane (15 mL) washed three times with water (5 mL), dried (Na₂SO₄), filtered and concentrated to afford 0.50 g (41% yield) of the title compound as an oil. ¹H-NMR (300 MHz, CDCl₃): δ 0.99 (t, 3H), 1.40-1.48 (m, 5H), 1.67 (m, 2H), 2.09 (m, 4H), 2.80-2.60 (m, 8H), 3.53 (m, 2H), 4.01 (q, 1H), 5.31-5.47 (m, 10H); MS (electrospray): 359.2 [M-H]⁻.

### Example 8: Preparation of tert-butyl 2-((5Z,8Z,11Z,14Z,17Z)-icosa-5,8,11,14,17-pentaenyloxy)-2-methylpropanoate

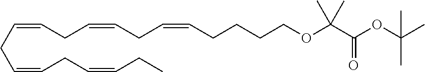 [SMILES: CC/C=C\C/C=C\C/C=C\C/C=C\C/C=C\CCCCOC(C)(C)C(=O)OC(C)(C)C]

A mixture of (5Z,8Z,11Z,14Z,17Z)-icosa-5,8,11,14,17-pentaen-1-ol, (0.83 g, 3.14 mmol), tetrabutylammonium chloride (0.24 g, 0.85 mmol) and t-butyl 2-bromo isobutyrate (3.50 g, 15.7 mmol) was dissolved in toluene (15 mL) and placed under nitrogen. An aqueous solution of sodium hydroxide (50%, 5 mL) was added slowly under vigorous stirring at room temperature. The resulting mixture was heated to 60° C. and stirred for six hours. The mixture was cooled, added water and extracted three times with ether. The combined organic extract was washed with brine, dried (Na₂SO₄), filtered and concentrated. The residue was purified by flash chromatography on silica gel using a gradient of 5-10% ethyl acetate in heptane as eluent. Concentration of the appropriate fractions afforded 0.60 g (44% yield) of the title compound as an oil. MS (electrospray): 453.3 [M+Na]⁺.

### Example 9: Preparation of 2-((5Z,8Z,11Z,14Z,17Z)-icosa-5,8,11,14,17-pentaenyloxy)-2-methylpropanoic acid

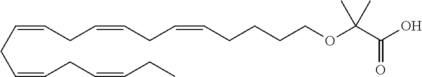 [SMILES: CC/C=C\C/C=C\C/C=C\C/C=C\C/C=C\CCCCOC(C)(C)C(=O)O]

Trifluoroacetic acid (5 mL) was added to a solution of tert-butyl 2-((5Z,8Z,11Z,14Z,17Z)-icosa-5,8,11,14,17-pentaenyloxy)-2-methylpropanoic (600 mg, 1.39 mmol) in dichloromethane (20 mL) under nitrogen and the reaction mixture was stirred at room temperature for two hours. Water was added and the aqueous phase was extracted twice with dichloromethane. The combined organic extract was washed with brine, dried (Na₂SO₄), filtered and concentrated. The residue was purified by flash chromatography on silica gel using a mixture of heptane, ethyl acetate and formic acid (80:20:1) as eluent. The appropriate fractions were concentrated and the residue (135 mg) was purified further by flash chromatography on silica gel using a gradient of 5-10% of a mixture of ethyl acetate and formic acid (95:5) in heptane as eluent. Concentration of the appropriate fractions afforded 80 mg slightly impure product. This material was dissolved in heptane (5 mL), washed twice with water (5 mL), dried (Na₂SO₄), filtered and concentrated to afford 40 mg (8% yield) of the title compound as an oil. ¹H-NMR (300 MHz, CDCl₃): δ 0.99 (t, 3H), 1.47 (s, 6H), 1.64 (m, 2H), 2.07 (m, 4H), 2.81-2.88 (m, 8H), 3.46 (t, 2H), 5.29-5.44 (m, 10H); MS (electrospray): 373.3 [M-H]⁻.

Example 10: Preparation of tert-butyl 2-ethyl-2-((5Z,8Z,11Z,14Z,17Z)-icosa-5,8,11,14,17-pentaen-1-yloxy)butanoate

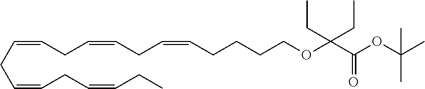 [SMILES: CC/C=C\C/C=C\C/C=C\C/C=C\C/C=C\CCCCOC(CC)(CC)C(=O)OC(C)(C)C]

tert-Butyl 2-((5Z,8Z,11Z,14Z,17Z)-icosa-5,8,11,14,17-pentaen-1-yloxy)butanoate (480 mg, 1.11 mmol) was added dropwise over 30 minutes to a solution of lithium diisopropylamine (LOA) (2.0 M, 750 μL, 1.50 mmol) in dry tetrahydrofuran (10 mL) held at −70° C. under nitrogen. The reaction mixture was stirred for 30 minutes. Ethyl iodide (312 mg, 2.00 mmol) was added in one portion and the resulting mixture was warmed to ambient temperature during 1 hour. The reaction mixture was stirred at ambient temperature for 17 hours. The mixture was poured into saturated NH₄Cl (aq.) (50 mL) and extracted with heptane (2×50 mL). The combined organic phases was washed successively with brine (50 mL), 0.25 M HCl (50 mL) and brine (50 mL), dried (MgSO₄), filtered and concentrated. The residue was purified by flash chromatography on silica gel using increasingly polar mixtures of heptane and ethyl acetate (100:0 ->95:5) as eluent. Concentration of the appropriate fractions afforded 343 mg (67% yield) of the title compound as an oil. ¹H NMR (300 MHz, CDCI₃): δ 0.84 (t, 6H), 0.99 (td, 3H), 1.35-1.55 (m, 11H), 1.54-1.69 (m, 2H), 1.68-1.87 (m, 4H), 1.99-2.24 (m, 4H), 2.74-2.99 (m, 8H), 3.31 (t, 2H), 5.23-5.52 (m, 10H); MS (electrospray): 401.3 [M-1]⁻.

### Example 11: Preparation of 2-ethyl-2-((5Z,8Z,11Z,14Z,17Z)-icosa-5,8,11,14,17-pentaen-1-yloxy)butanoic acid

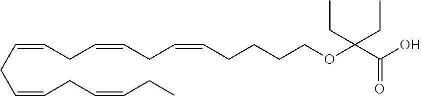 [SMILES: CC/C=C\C/C=C\C/C=C\C/C=C\C/C=C\CCCCOC(CC)(CC)C(=O)O]

A mixture of formic acid (5 ml) and teat-butyl 2-ethyl-2-((5Z,8Z,11Z,14Z,17Z)-icosa-5,8,11,14,17-pentaen-1-yloxy)butanoate (250 mg, 0.55 mmol) was stirred vigorously under nitrogen at room temperature for 4.5 hours. The formic acid was removed in vacuo. The residue was purified by flash chromatography on silica gel using increasingly polar mixtures of heptane and ethyl acetate (100:0->80:20) as eluent. Concentration of the appropriate fractions afforded 163 mg (74% yield) of the title compound as an oil. ¹H NMR (300 MHz, CDCl₃): δ 0.86 (t, 6H), 0.99 (t, 3H), 1.36-1.57 (m, 2H), 1.68 (dd, 2H), 1.73-1.98 (m, 4H), 2.11 (tt, 4H), 2.70-3.01 (m, 8H), 3.39 (t, 2H), 5.20-5.56 (m, 10H). MS (electrospray): 481.4 [M+Na]⁺.

### Example 12: Preparation of ethyl 2-((5Z,8Z,11Z,14Z,17Z)-icosa-5,8,11,14,17-pentaen-1-yloxy)butanoate

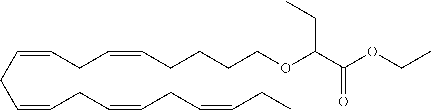 [SMILES: CC/C=C\C/C=C\C/C=C\C/C=C\C/C=C\CCCCOC(CC)C(=O)OCC]

Dicyclohexylmethanediimine (DCC) (304 mg, 1.47 mmol) and N,N-dimethylpyridin-4-amine (DMAP) (10 mg, 0.067 mmol) were added to a stirred solution of 2-(((5Z,8Z,11Z,14Z,17Z)-icosa-5,8,11,14,17-pentaen-1-yl)oxy)butanoic acid (501.3 mg, 1.335 mmol) in dichloromethane (DCM) (4 mL) at 0 ° C. under N₂-atmosphere. The mixture was stirred for 5 minutes, before ethanol (EtOH) (0.16 mL, 2.67 mmol) was added. The resulting mixture was stirred at room temperature for 20 hours. The reaction mixture was purified by flash chromatography on silica gel using increasingly mixtures of heptane and ethyl acetate (100:0→99:1) as eluent. Concentration of the appropriate fractions afforded 473 mg (88% yield) of the title compound as an oil. ¹H NMR (300 MHz, chloroform-d) δ 0.95 (2×t, 6H), 1.37-1.48 (m, 2H), 1.54-1.79 (m, 4H), 2.01-2.10 (m, 4H), 2.77-2.84 (m, 8H), 3.27-3.34 (m, 1H), 3.53-3.60 (m, 1H), 3.69-3.73 (dd, 1H), 4.13-4.24 (m, 2H), 5.25-5.33 (m, 10H), MS (electrospray); 425.3 [M+Na]⁺; HRMS (electrospray): Found 425.3021 [M+Na]⁺, calcd. for [C₂₆H₄₂O₃+Na]⁺425.3031.

### Example 13: Preparation of isopropyl 2-(((5Z,8Z,11Z,14Z,17Z)-icosa-5,8,11,14,17-pentaen-1-yl)oxy)butanoate

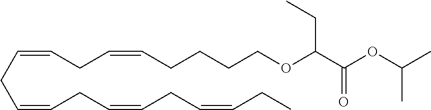 [SMILES: CC/C=C\C/C=C\C/C=C\C/C=C\C/C=C\CCCCOC(CC)C(=O)OC(C)C]

DCC (310 mg, 1.47 mmol) and DMAP (9 mg, 0.067 mmol) were added to a stirred solution of 2-(((5Z,8Z,11Z,14Z,17Z)-icosa-5,8,11,14,17-pentaen-1-yloxy)butanoic acid (501 mg, 1.335 mmol) in DCM (4 mL) at 0° C. under N₂-atmosphere. The mixture was stirred for 5 minutes, before isopropanol (iPrOH) (0.16 mL, 2.67 mmol) was added. The resulting mixture was stirred at room temperature for 20 hours. The mixture was filtered and concentrated in vacuo. The residue was added heptane (50 mL), filtered and concentrated in vacuo. The residue was purified by flash chromatography on silica gel using 1% ethyl acetate in heptane as eluent. Concentration of the appropriate fractions afforded 496 mg (89% yield) of the title compound as an oil. ¹H NMR (300 MHz, CDCl₃): δ 0.97 (2×t, 6H), 1.25 (m, 6H), 1.42-1.50 (m, 2H)), 1.61-1.70 (m, 2H), 1.70-1.77 (m, 2H), 2.05-2.12 (m, 4H), 2,79-2.86 (m, 8H), 3.29-3.34 (m, 1H), 3.54-3.59 (m, 1H), 3.67-3.71 (m, 1H), 5.06-5.10 (m, 1H), 5.31-5.42 (m, 10H); MS (electrospray): 439.3 [MH+Na]⁺.

### Example 14: Preparation of methyl 2-(((5Z,8Z,11Z,14Z,17Z)-icosa-5,8,11,14,17-pentaen-1-yl)oxy)butanoate

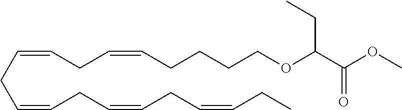 [SMILES: CC/C=C\C/C=C\C/C=C\C/C=C\C/C=C\CCCCOC(CC)C(=O)OC]

Sulphuric acid (0.049 ml, 0.918 mmol) was added to a solution of 2-(((5Z,8Z,11Z,14Z,17Z)-icosa-5,8,11,14,17-pentaen-1-yl)oxy)butanoic acid ((385 mg, 1.028 mmol) in methanol (20 ml) at room temperature under N₂-atmosphere and the resulting mixture was stirred at room temperature overnight. MS (electrospray): 389.3 [M+1]⁺.

### Example 15: Preparation of butyl 2-(((5Z,8Z,11Z,14Z,17Z)-icosa-5,8,11,14,17-pentaen-1-yl)oxy)butanoate

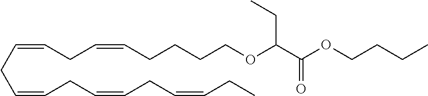 [SMILES: CC/C=C\C/C=C\C/C=C\C/C=C\C/C=C\CCCCOC(CC)C(=O)OCCCC]

Sulphuric acid (0.049 ml, 0.918 mmoL) was added to a solution of 2-(((5Z,8Z,11Z,14Z,17Z)-icosa-5,8,11,14,17-pentaen-1-yl)oxy)butanoic acid ((33 g, 88 mmol) butan-1-ol (400 mL, 4.37 mmol) at room temperature under N₂-atmosphere and the reaction mixture was stirred for 120 hours. Heptane (400 mL) and ethyl acetate (400 mL) was added, and the solution was washed with saturated aq. NaHCO₃ (3×300 mL) and water (2×300 mL). The combined aqueous phase was extracted with heptane/ether (1:1) (2×300 mL). The combined organic phase was dried (Na₂SO₄), filtered and concentrated in vacuo. The residue was purified by flash chromatography using increasingly mixtures of heptane and ethyl acetate (99:1→96:4) as eluent. Concentration of the appropriate fractions afforded 26.3 g (67% yield) of title compound as an oil. ¹H NMR (400 MHz, CDCl₃) δ 0.93-1.02 (m, 9H), 1.36-1.51 (m, 4H), 1.60-1.70 (m, 4H), 1.72-1.84 (m, 2H), 2.05-2.16 (m,4H), 2.78-2.92 (m, 8H), 3.28-339 (m, 1H), 3.54-3.65 (m, 1H), 3.70-3.82 (m, 1H), 4.08-4.24 (m, 2H), 5.27-5.48 (m, 10H), MS (electrospray): 453.2 [M+Na]⁺.

### Example 16: Preparation of 2,3-dihydroxypropyl 2-(((5Z,8Z,11Z,14Z,17Z)-icosa-5,8,11,14,17-pentaen-1-yloxy)butanoate

### Step a) Preparation (2,2-dimethyl-1,3-dioxolan-4-yl)methyl 2-(((5Z,8Z,11Z,14Z,17Z)-icosa-5,8,11,14,17-pentaen-1-yloxy)butanoate

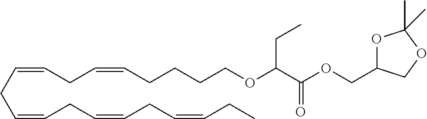 [SMILES: CC/C=C\C/C=C\C/C=C\C/C=C\C/C=C\CCCCOC(CC)C(=O)OCC1COC(C)(C)O1]

2-(((5Z,8Z,11Z,14Z,17Z)-icosa-5,8,11,14,17-pentaen-1-yl)butanoic acid (25 g, 66.7 mmol) and DMAP (8.15 g, 66.7 mmol) were added to a solution of 2,2-dimethyl-1,3-dioxolane-4-methanol (7.54 60.7 mmol) in chloroform (150 mL) under nitrogen atmosphere. A solution of DCC (13.77 g, 66.7 mmol) in chloroform (65 mL) was then added drop wise at ambient temperature. The mixture was stirred overnight and concentrated in vacuo. The residue was purified by flash chromatography on silica gel using increasingly polar mixtures of 10% ethyl acetate in heptane as eluent. Concentration of the appropriate fractions afforded 19.6 g (66% yield) of the title product as an oil. ¹H NMR (300 MHz, CDCl₃) δ 0.99 (t, 6H), 1.37-1.40 (m, 3H), 1.41-1.53 (m, 5H), 1.59-1.71 (m, 2H), 1.72-1.85 (m, 2H), 2.05-2.14 (m, 4H), 2.74-2.95 (m, 8H), 3.31-3.38 (m, 1H), 3.57-3.65 (m, 1H), 3.72-3.86 (m, 2H), 4.07-4.12 (m, 1H), 4.15-4.27 (m, 2H), 4.29-4.40 (m, 1H), 5.23-5.50 (m, 10H). MS (electrospray): 511.3 [M+Na]⁺.

### Step b) Preparation of 2,3-dihydroxypropyl 2-(((5Z,8Z,11Z,14Z, 17Z)-icosa-5,8,11,14,17-pentaen-1-yl)oxy)butanoate

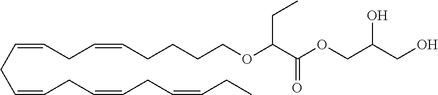 [SMILES: CC/C=C\C/C=C\C/C=C\C/C=C\C/C=C\CCCCOC(CC)C(=O)OCC(O)CO]

To a solution of (2,2-dimethyl-1,3-dioxolan-4-yl)methyl 2-(((5Z,8Z,11Z,14Z,17Z)-icosa-5,8,11,14,17-pentaen-1-yl)oxy)butanoate (27.5 g, 56.3 mmol) in dioxane (280 mL) at room temperature under nitrogen was added aq. HCl (37% (w/w), 28 mL, 341 mmol) and the mixture was stirred for 60 minutes. The mixture was then carefully poured into sat, aq. NaHCO₃ (500 mL) and extracted with EtOAc (2×300). The organic phase was washed with 1M HCl (200 mL), brine (200 mL), dried (Na₂SO₄), filtered and concentrated in vacuo. The residue was purified by flash chromatography on silica gel using heptane and ethyl acetate (50:50) as eluent. Concentration of the appropriate fractions afforded 19 g of the title product as an oil, contaminated with ˜10% of the isomer 1,3-dihydroxypropan-2-yl 2-(((5Z,8Z,11Z,14Z,17Z)-icosa-5,8, 11,14,17-pentaen-1-yl)oxy)butanoate. The material was mixed with 1.35 gram of another hatch, before further purified by preparative HPLC. An isocratic 17:83 mixture of water/acetonitrile (9:1) to acetonitrile (100%) was used as eluent. Concentration of the appropriate fractions afforded 11.3 g (38% yield) of the title product as an oil. ¹H NMR (300 MHz, CDCl₃) δ 0.97-1.03 (m, 6H), 1.41-1.51 (m, 2H), 1.59-1.69 (m, 2H), 1.72-1.87 (m, 2H), 2.05-2.14 (m, 5H), 2.56 (s, 1H), 2.73-2.94 (m, 8H), 3.33-3.40 (m, 1H), 3.55-3.68 (m, 2H), 3.69-3.77 (m, 1H), 3.79-3.85 (m, 1H), 3.93-4.03 (m, 1H), 4.15-4.37 (m, 2H), 5.25-5.51 (m, 10H). MS (electrospray): 471.3 [M+Na]⁺.

Example 17: Preparation of 1,3-dihydroxypropan-2-yl 2-(((5Z,8Z,11Z,14Z,17Z)-icosa-5,8,11,14,17-pentaen-1-yloxy)butanoate

### Step a) Preparation of oxiran-2-ylmethyl 2-(((5Z,8Z,11Z,14Z,17Z)-icosa-5,8,11,14,17-pentaen-1-yl)oxy)butanoate

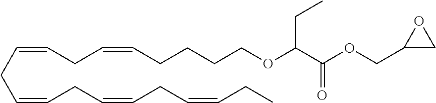 [SMILES: CC/C=C\C/C=C\C/C=C\C/C=C\C/C=C\CCCCOC(CC)C(=O)OCC1CO1]

A mixture of 2-(((5Z,8Z,11Z,14Z,17Z)-icosa-5,8,11,14,17-pentaen-1-yl)oxy)butanoic acid (800 mg, 2.14 mmol), glycidol (0.17 mL, 2.56 mmol). 1-(3-dimethylaminopropyl)-3-ethylcarbodiimide hydrochloride (EDC*HCl) (491 mg, 2.56 mmoL) and 4-dimethylaminopyridine (DHAP) (313 mg, 2.56 mmol) in dry DCM (7 mL) was stirred at room temperature under N₂-atmosphere. The reaction mixture was concentrated in vacuo. The residue was purified by flash chromatography on silica gel using increasingly polar mixtures of heptane and ethyl acetate (99:1→95:5) as eluent. Concentration of the appropriate fractions afforded 527 mg (57% yield) of the title product as an oil. ¹H NMR (400 MHz, CDCl₃) δ 0.94-0.98 (m, 6H), 1.40-1.44 (m, 2H), 1.57-1.64 (m, 2H), 1.70-1.82 (m, 2H), 2.02-2.12 (m, 4H), 2.63 (bs, 1H), 2.78-2.84 (m, 9H), 3.20 (bs, 1H), 3.30-3.35 (m, 1H), 3.55-3.61 (m, 1H), 3.77-3.80 (m, 1H), 3.94-4.01 (m, 1H), 4.42-4.48 (m, 1H), 5.36-5.26 (nn, 10H). MS (electrospray): 453.3 [M+Na]⁺.

### Step b) Preparation of 2-((2-(((5Z,8Z,11Z,14Z,17Z)-icosa-5,8,11,14,17-pentaen-1-yl)oxy)butanoyl)oxy)propane-1,3-diyl bis(2,2,2-trifluoroacetate)

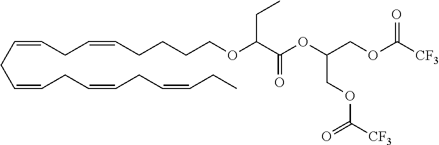 [SMILES: CC/C=C\C/C=C\C/C=C\C/C=C\C/C=C\CCCCOC(CC)C(=O)OC(COC(=O)C(F)(F)F)COC(=O)C(F)(F)F]

Trifluoroacetic anhydride (TFAA) (0.55 mL, 3.96 mmoL) in dry DCM (3 mL) was added portion wise to a precooled solution of oxiran-2-ylmethyl 2-(((5Z,8Z,11Z,14Z,17Z)-icosa-5,8,11,14,17-pentaen-1-yloxy)butanoate (286 mg, 0.66 mmoL) in dry DCM (3 mL) at −20° C. under N₂-atmosphere. The cooling bath was removed and the mixture was stirred for 19 hours at ambient temperature, before reaction mixture was concentrated in vacuo pressure. The residue was dissolved in toluene (6 mL) and passed through a pad of silica (6.5 g) eluting with toluene (150 mL). Concentration in vacuo to afforded 357 mg (84% yield) of the title compound as an oil. ¹H NMR (400 MHz, CDCl₃) δ 0.95 (2×t, 6H), 1.38-1.45 (m, 2H), 1.57-1.63 (m, 2H), 1.66-1.78 (m, 2H), 2.09-2.02 (m, 4H), 2.78- 2.84 (m, 8H), 3.27-3.33 (m, 1H), 3.51-3.56 (m, 1H), 3.77 (dd, 1H), 4.30-4.53 (m, 2H), 4.60-4.69 (m, 2H), 5.17-5.43 (m, 10H), 5.43-5.55 (m, 1H). MS (electrospray): 661.1 [M+Na]⁺.

### Step c) Preparation of 1,3-dihydroxypropan-2-yl 2-(((5Z,8Z,11Z,14Z,17Z)-icosa-5,8,11,14,17-pentaen-1-yl)oxy)butanoate

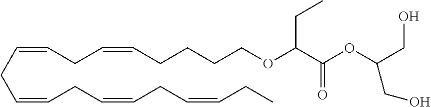 [SMILES: CC/C=C\C/C=C\C/C=C\C/C=C\C/C=C\CCCCOC(CC)C(=O)OC(CO)CO]

A solution of pyridine (0.4 mL, 4.95 mmol) and methanol (0.3 mL, 7.41 mmol) in pentane/DCM (2:1) (4.5 mL) was added drop wise to a solution of 2-((2-(((5Z,8Z,11Z,14Z,17Z)-icosa-5,8,11,14,17-pentaen-1-yl)oxy)butanoyl)oxy)propane-1,3-diyl bis(2,2,2-trifluoroacetate) (354 mg, 0.552 mmol) in pentane/DCM (2:1) (5 mL) cooled to −20° C. under N₂-atmosphere. The cooling bath was removed and the mixture was stirred for 3 hours at room temperature, before concentrated in vacuo. The residue was purified by flash chromatography on silica gel using increasingly polar mixtures of heptane and ethyl acetate (95:5→90:10→80:20→50:50) as eluent. Concentration of the appropriate fractions afforded 223 mg of the title product as crude oil. Purification by preparative HPLC afforded 58 mg (22% yield) of the title compound as an oil. ¹H NMR (400 MHz, CDCl₃) δ 0.95 (t, 3H), 0.96 (t, 3H), 1.38-1.45 (m, 2H), 1.54-1.64 (m, 2H), 1.67-1.84 (m, 2H), 2.01-2.09 (m, 4H), 2.45 (bs, 2H), 2.83-2.77 (m, 8H), 3.36-3.30 (m, 1H), 3.60-3.55 (m, 1H), 3.84-3.78 (m, 5H), 4.98-4.93 (m, 1H), 5.65-5.09 (m, 10H). MS (electrospray): 471.1 [M+Na]⁺.

### Example 18: Preparation of 3-hydroxypropane-1,2-diyl-1,2-diyl bis(2-(((5Z,8Z,11Z,14Z,17Z)-icosa-5,8,11,14,17-pentaen-1-yl)oxy)butanoate)

### Step a) Preparation of tert-butyl((2,2-dimethyl-1,3-dioxolan-4-yl)methoxy)dimethylsilane

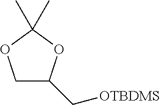 [SMILES: CC1(C)OCC(CO[Si](C)(C)C(C)(C)C)O1]

tert-Butyl-chlorodimethylsilane (14.41 g, 91 mmol) was added to a solution of (2,2-dimethyl-1,3-dioxolan-4-yl)methanol (10 g, 76 mmol) and imidazole (7.73 g, 114 mmol) in THF (100 mL) at ambient temperature under nitrogen atmosphere. The mixture was stirred for 1.5 hours, poured into water (200 mL) and extracted with tert-butyl methyl ether (2×150 mL). The phases were separated and the organic layer was washed with water (100 brine (100 mL), dried (Na₂SO₄), filtered and concentrated in vacuo. The residue was purified by flash chromatography on silica gel using 3% ethyl acetate in heptane as eluent. Concentration of the appropriate fractions afforded 18 g (97% yield) of the title compound as an oil. ¹H NMR (300 MHz, CDCl₃) δ 0.02-0.05 (m, 6H), 0.85-0.89 (m, 9H), 1.31-1.35 (m, 3H), 1.35-1.40 (m, 3H), 3.50-3.60 (m, 1H), 3.63-3.72 (m, 1H), 3.75-3.85 (m, 1H), 3.96-4.05 (m, 1H), 4.07-4.18 (m, 1H). MS (electrospray): 229.2 [M+Na]⁺.

### Step b) Preparation of 3-((tert-butyldimethylsilyl)oxy)propane-1,2-diol

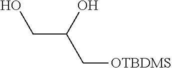 [SMILES: CC(C)(C)[Si](C)(C)OCC(O)CO]

To a solution of tert-butyl((2,2-dimethyl-1,3-dioxolan-4-yl)methoxy)dimethylsilane in chloroform (60 mL) was added FeCl₃×6H₂O absorbed on silica gel (30 g, 11.9 mmol) and the mixture was stirred overnight. The mixture was filtered and concentrated in vacuo. The residue was purified by flash chromatography on silica gel using increasingly polar mixtures of heptane and ethyl acetate (50:50→25:75) as eluent. Concentration of the appropriate fractions afforded 760 mg (9% yield) of the title compound as an oil. ¹H NMR (300 MHz, CDCl₃) δ 0.09-0.12 (m, 6H). 0.91- 0.95 (m, 9H), 2.11-2.17 (m, 1H), 2.60 (d, 1H), 3.57- 3.85 (m, 5H). MS (electrospray): 229.2 [M+Na]⁺.

### Step c) Preparation of 3-((tert-butyldimethylsilyl(oxy)propane-1,2-diyl bis(2-(((5Z,8Z,11Z,14Z,17Z)-icosa-5,8,11,14,17-pentaen-1-yl)oxy)butanoate)

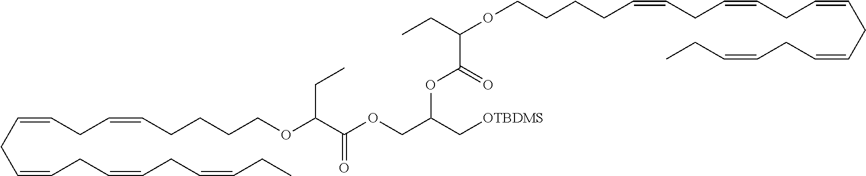 [SMILES: CC/C=C\C/C=C\C/C=C\C/C=C\C/C=C\CCCCOC(CC)C(=O)OCC(CO[Si](C)(C)C(C)(C)C)OC(=O)C(CC)OCCCC/C=C\C/C=C\C/C=C\C/C=C\C/C=C\CC]

To a solution of 3-((tert-butyldimethylsilyl)oxy)propane-1,2-diol (0.91 g, 4.41 mmol) in DMF (20 ml) under N₂-atmosphere at ambient temperature were added 2-(((5Z,8Z,11Z,14Z,17Z)-icosa-5,8,11,14,17-pentaen-1-yl)oxy)butanoic acid (3.47 g, 9.3 mmol), DMAP (1.13 g, 9.3 mmol), 1-(3-dimethylaminopropyl)-3-ethylcarbodiimide hydrochloride (DCI) (1.776 g, 9.26 mmol) and dry DCM (60 ml). The mixture was stirred overnight, before the reaction mixture was diluted with diethyl ether (200 mL). The mixture was washed with 1M HCl (100 mL) and brine (100 dried (Na₂SO₄), filtered and concentrated in vacuo. The residue was purified by flash chromatography on silica gel using 3% ethyl acetate in heptane as eluent. Concentration of the appropriate fractions afforded 2.26 g (56% yield) of the title compound as an oil. ¹H NMR (300 MHz, CDCl₃) δ 0.08 (s, 6H), 0.90 (d, 9H), 0.95-1.03 (m, 12H), 1.40-1.52 (m, 4H), 1.58-1.69 (m, 4H), 1.70-1.83 (m, 4H), 2.04-2.15 (m, 8H), 2.77-2.92 (m, 16H), 3.27-3.37 (m, 2H), 3.57-3.67 (m, 2H), 3.72-3.80 (m, 4H), 4.14-4.32 (m, 1H), 4.41-4.56 (m, 1H), 5.09-5.22 (m, 1H), 5.23-5.49 (m, 20H). MS (electrospray): 941.6 [M+Na]⁺.

### Step d) Preparation of 3-hydroxypropane-1,2-diyl bis(2-(((5Z,8Z,11Z,14Z,17Z)-icosa-5,8,11,14,17-pentaen-1-yl)oxy)butanoate)

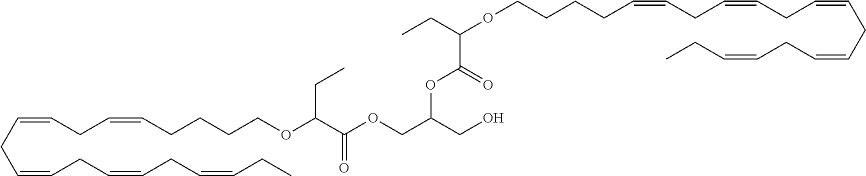 [SMILES: CC/C=C\C/C=C\C/C=C\C/C=C\C/C=C\CCCCOC(CC)C(=O)OCC(CO)OC(=O)C(CC)OCCCC/C=C\C/C=C\C/C=C\C/C=C\C/C=C\CC]

To a solution of 3-((tert-butyldimethylsilyl)oxy)propane-1,2-diyl bis(2-(((5Z,8Z,11Z,14Z,17Z)-icosa-5,8,11,14,17-pentaen-1-yl)oxy)butanoate) (2.26 g, 2.46 mmol) in dioxane (100 mL) was added aq, HCl (37% (w/w, 2 mL) and the mixture was stirred for 3 hours under nitrogen atmosphere at ambient temperature, before concentrated in vacuo. The residue was purified by flash chromatography on silica gel using 15% ethyl acetate in heptane as eluent. Concentration of the appropriate fractions afforded 0.83 g (42% yield) of the title compound as an oil. ¹H NMR (300 MHz, CDCl₃) δ 0.96-1.03 (m, 12H), 1.40-1.53 (m, 4H), 1.58-1.68 (m, 4H), 1.70-1.85 (m, 4H), 1.87-2.01 (m, 1H), 2.05-2.15 (m, 8H), 2.75-2.95 (m, 16H), 3.28-3.41 (m, 2H), 3.56-3.65 (m, 2H), 3.73-3.85 (m, 4H), 4.24-4.37 (m, 1H), 4.42-4.53 (m, 1H), 5.14-5.23 (m, 1H), 5.26-5.51 (m, 20H). MS (electrospray): 827.5 [M+Na]⁺.

### Example 19: Preparation of 2-hydroxypropane-1,3-diyl bis(2-(((5Z,8Z,11Z,14Z,17Z)-icosa-5,8,11,14,17-pentaen-1-yl)oxy)butanoate)

### Step a) 2-oxopropane-1,3-diyl bis(2-(((5Z,8Z,11Z,14Z,17Z)-icosa-5,8,11,14,17-pentaen-1-yl)oxy)butanoate)

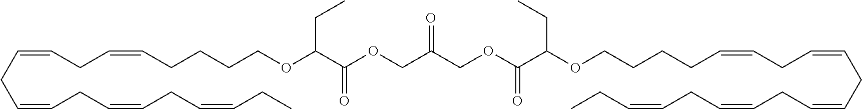 [SMILES: CC/C=C\C/C=C\C/C=C\C/C=C\C/C=C\CCCCOC(CC)C(=O)OCC(=O)COC(=O)C(CC)OCCCC/C=C\C/C=C\C/C=C\C/C=C\C/C=C\CC]

2-(((5Z,8Z,11Z,14Z,17Z)-icosa-5,8,11,14,17-pentaen-1-yl)oxy)butanoic acid (5.0 g, 13.4 mmol) and DMAP (1.63 g, 13.4 mmol) were added to a solution of 1,3-dihydroxyacetone dimer (1.145 g, 6.36 mmol) in chloroform (25 mL) under nitrogen atmosphere. A solution of DCC (2.75 g, 13.35 mmol) in chloroform (10 mL) was then added drop wise at ambient temperature. The mixture was stirred overnight at room temperature, before concentrated in vacuo. The residue was purified by flash chromatography on silica gel using increasingly polar mixtures of heptane and ethyl acetate (90:10→88:12) as eluent. Concentration of the appropriate fractions afforded 2.4 g (47% yield) of the title compound as an oil. ¹H NMR (300 MHz, CDCl₃) δ 0.97-1.06 (m, 12H). 1.38-1.53 (m, 4H), 1.57-1.73 (m, 4H), 1.73-1.96 (m, 4H), 2.03-2.17 (m, 8H), 2.76-2.92 (m, 16H), 3.35-3.42 (m, 2H), 3.63-3.70 (m, 2H), 3.89 (dd, 2H), 4.75-4.93 (m, 4H), 5.27-5.49 (m, 20H). MS (electrospray): 827.5 [M+Na]⁺.

### Step b) 2-hydroxypropane-1,3-diyl bis(2-(((5Z,8Z,11Z,14Z,17Z)-icosa-5,8,11,14,17-pentaen-1-yl )oxy)butanoate)

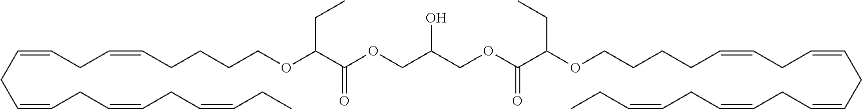 [SMILES: CC/C=C\C/C=C\C/C=C\C/C=C\C/C=C\CCCCOC(CC)C(=O)OCC(O)COC(=O)C(CC)OCCCC/C=C\C/C=C\C/C=C\C/C=C\C/C=C\CC]

NaBH₄ (0.336 g, 8.87 mmol) was added carefully to a solution of 2-oxopropane-1,3-diyl bis(2-(((5Z,8Z,11Z,14Z,17Z)-icosa-5,8,11,14,17-pentaen-1-yl)oxy)butanoate) (3.24 g, 4.03 mmol) in THF (55 mL) and water (4 mL) at 0° C. The mixture was stirred for 15 minutes at 0° C. Acetic acid (1 mL) was then added carefully followed by ethyl acetate (100 mL). The mixture was washed with water (100 mL), saturated aq. NaHCO₃ (100 mL) and brine, before dried (Na₂SO₄), filtered and concentrated in vacuo. The residue was combined with another hatch of the material before purified by flash chromatography on silica gel using 15% ethyl acetate in heptane as eluent. Concentration of the appropriate fractions afforded 1.62 g (50% yield) of the title compound as an oil. ¹H NMR (300 MHz, CDCl₃) δ 0.97-1.03 (m, 12H), 1.41-1.52 (m, 4H), 1.58-1.69 (m, 6H), 1.71-1.87 (m, 4H), 2.05-2.14 (m, 8H), 2.38-2.42 (m, 1H), 2.78-2.92 (m, 16H), 3.32-3.39 (m, 2H), 3.57-3.64 (m, 2H), 3.80-3.84 (m, 2H), 4.05-4.34 (m, 5H), 5.26-5.49 (m, 2H). MS (electrospray): 827.5 [M+Na]⁺.

### Example 20: Preparation of propane-1,2,3-triyl tris(2-(((5Z,8Z,11Z,14Z,17Z-icosa-5,8,11,14,17-pentaen-1-yl)oxy)butanoate)

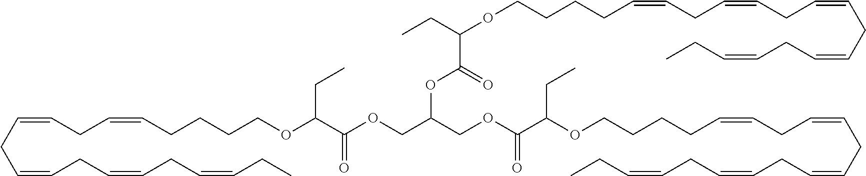 [SMILES: CC/C=C\C/C=C\C/C=C\C/C=C\C/C=C\CCCCOC(CC)C(=O)OCC(COC(=O)C(CC)OCCCC/C=C\C/C=C\C/C=C\C/C=C\C/C=C\CC)OC(=O)C(CC)OCCCC/C=C\C/C=C\C/C=C\C/C=C\C/C=C\CC]

2-(((5Z,8Z,11Z,14Z,17Z)-icosa-5,8,11,14,17-pentaen-1-yl)oxy)butanoic acid (4.0 g, 10.7 mmol), 4-dimethylaminopyridine (1.305 g, 10.7 mmol), 1-(3-dimethylaminopropyl)-3-ethylcarbodiimide hydrochloride (2.047 g, 10.7 mmol) and dry DCM (30 ml) was added to a solution of glycerol (0.173 ml, 2.373 mmol) in DMF (10 ml) under N₂-atmosphere at room temperature. The mixture was stirred overnight, before the reaction mixture was diluted with diethyl ether (250 mL). The mixture was washed with aq. 1M HCl (100 mL) and brine (100 mL), before dried (Na₂SO₄), filtered and evaporated in vacuo. The residue was purified by flash chromatography on silica gel using 5% ethyl acetate in heptane as eluent. Concentration of the appropriate fractions afforded 2.1 g (73% yield) of the title compound as an oil. ¹H NMR (300 MHz, CDCl₃) δ 0.91-1.05 (m, 18H), 1.40-1.52 (m, 6H), 1.57-1.69 (m, 6H), 1.69-1.86 (m, 6H), 2.01-2.17 (m, 12H), 2.69-2.96 (m, 24H), 3.27-3.38 (m, 3H), 3.53-3.67 (m, 3H), 3.73-3.81 (m, 3H), 4.17-4.27 (m, 2H), 4.37-4.54 (m, 2H), 5.28-5.47 (m, 30H). MS (electrospray): 1183.8 [M+Na]⁺.

### Example 21: Preparation of calcium 2-(((5Z,8Z,11Z,14Z,17Z)-icosa-5,8,11,14,17-pentaen-1-yl)oxy)butanoate

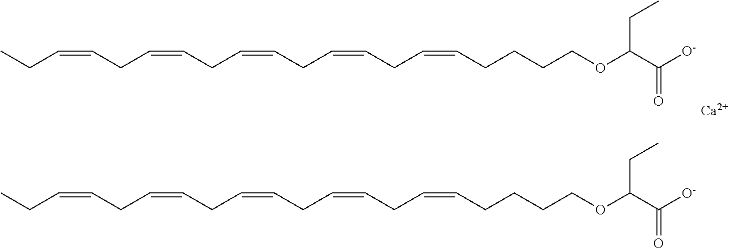 [SMILES: CC/C=C\C/C=C\C/C=C\C/C=C\C/C=C\CCCCOC(CC)C(=O)[O-] ; [Ca+2] ; CC/C=C\C/C=C\C/C=C\C/C=C\C/C=C\CCCCOC(CC)C(=O)[O-]]

2-(((5Z,8Z,11Z,14Z,17Z)-icosa-5,8,11,14,17-pentaen-1-yl)oxy)butanoic acid (1.87 g, 4.99 mmol, 93%) was mixed with CaCO₃ (0.25 g, 2.50 mmol). Water (1 mL) was added and the mixture was stirred with mechanical stirring at RT for 1 hour. CO₂ develops. Dense and homogeneous pasta was formed. With stirring, acetone (7 ml) was added. A solid materiel separates. The solid materiel was filtered of and dried over nitrogen sealed and stored in the fridge at 4° C. Yield: 1.86 grams (95%). The solid was not further characterized by analytical or spectroscopic methods, but a few experiments indicating that the calcium salt has formed was performed:

The solid materiel melts on a hot plate below 100° C. No sharp melting point was determined

The material do not liberate CO₂ on addition of acid, but “dissolves” and precipitates as an oil

### Example 22: Preparation of sodium 2-(((5Z,8Z,11Z,14Z,17Z)-icosa-5,8,11,14,17-pentaen-1-yl)oxy)butanoate

2-(((5Z,8Z,11Z,14Z,17Z)-icosa-5,8,11,14,17-pentaen-1-yl)oxy)butanoic acid (1.87 g. 4.99 mmol, 93%) was mixed with NaHCO₃ (0.420 g, 5.00 mmol). Water (1 mL) was added and the mixture was stirred with mechanical stirring at RT for 1 hour. CO₂ develops, and a thick homogeneous pasta was formed. With stirring, ethanol (7 ml) was added to the reaction flask. The sodium salt formed from 2-(((5Z,8Z,11Z,14Z,17Z)-Icosa-5,8,11,14,17-pentaen-1-yloxy)butanoic acid goes into solution upon addition of ethanol (7 mL). Small amounts of unreacted NaHCO₃ was filtered of and the solution was evaporated to dryness. The crude slightly viscous oil was evaporated two times with 96% ethanol to remove traces of water.

### Example 23: Preparation of 2-hydroxy-N,N,N-trimethylethan-1-aminium 2-(((5Z,8Z,11Z,14Z,17Z)-icosa-5,8,11,14,17-pentaen-1-yloxy)butanoate

Choline hydroxide (327.7 μL) in water was pipetted into a scintillation vial with ca. 2.5mL MTBE and 7.5 mL of n-Heptane. Within a nitrogen chamber, 2-(((5Z,8Z,11Z,14Z,17Z)-icosa-5,8,11,14,17-pentaen-1-yl)oxy)butanoic acid (500 mg, 95,8%) was transferred into the vial. Within a nitrogen chamber ca. 1.0 mL of water was added to the vial slowly and under stirring. The vial was then sealed. The reaction mixture was stirred for ca. 30 minutes. The formed 2-(((5Z,8Z,11Z,14Z,17Z)-icosa-5,8,11,14,17-pentaen-1-yl)oxy)butanoic acid choline salt was a rigid, gel-like material which was filtered on a Buchner funnel. The wet material on the filter was washed 3 times using 1 mL of MTBE. The washed material appeared as a rigid gel-like solid.

### Example 24 Pre-Clinical Study

### Evaluation of apoC-III Regulation in a Dyslipidemic Mouse Model (APOE*3Leiden Transgenic Mice)

The APOE*3Leiden transgenic mouse is expressing a variant of the human apolipoprotein E3 (APOE3), the APOE*3Leiden, in addition to the human apolipoprotein C1 (APOC1). The APOE*3Leiden transgenic mice exhibit elevated plasma cholesterol and triglyceride levels, mainly confined to the VLDL/LDL sized lipoprotein fraction (Van den Maagdenberg A M J M et al, Transgenic mice carrying the apolipoprotein E3-Leiden gene exhibit hyperlipoproteinemia, J Bial Chem 1993; 268: 10540-10545). In contrast to normal wild-type mice, the APOE*3Leiden transgenic mice are highly responsive to diet and hypolipidemic drugs affecting plasma VLDL and chylomicron levels (Van Vlijmen B et al, Diet-induced hyperlipoproteinemia and atherosclerosis in apolipoprotein E3-Leiden transgenic mice, J Clin Invest 1994; 93: 1403-1410; Groot PHE, et al. Quantitative assessment of aortic atherosclerosis in apoE3Leiden transgenic mice and its relationship to serum cholesterol exposure, Arterioscler Thromb Vase Biol 1996; 16: 926-933). Consequently, this model is appropriate to evaluate effects of lipid lowering drugs.

In this study, female APOE*3Leiden transgenic mice were put on a semi-synthetic Western-type diet (WTD; 15% cocoa butter, 40% sucrose and 0.25% cholesterol; all w/w). After 4 weeks with this diet the plasma cholesterol level reached mildly elevated levels of about 12-15 mmol/l. The mice were then sub-divided into groups of 10 mice each, matched for plasma cholesterol, triglycerides and body weight (t=0). The test substances were tested at 0.3 mmol/kg bw/day and were administered orally as admix to the WTD. After 4 weeks, all animals were sacrificed and serum and tissues were collected.

Liver tissues were stored in RNA later (Qiagen) at −80° C. Tissue was homogenized in RLT buffer with dithiothreitol (Qiagen) and RNA was isolated using the RNeasy kit (Qiagen), following the manufacturer's procedure. The quality of the isolated RNA was tested on a Bioanalyser (Agilent) showing RIN (RNA integrity number) values between 8.1 and 9.5 which indicates good quality, cDNA was synthesized by the “RNA to cDNA” kit (Applied Biosystems). Gene expression was measure using Low Density Arrays (LDA, specific for mouse RNA (Applied Biosystems)). Each sample was measured in 3 parallels, and the results are presented as the mean value relative to control (WTD without addition), The fold change in gene expression was calculated by the ΔΔCt method, using Rplp0 as housekeeping gene and the mean of the control samples as calibrator.

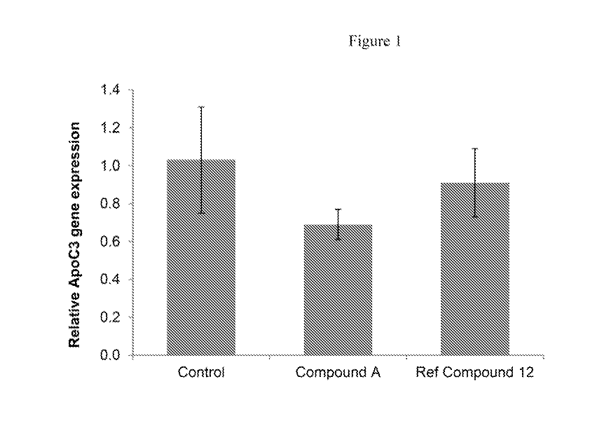

The results shown in FIG. 1 establish that mice fed Compound A (Example 2) have significantly lower apoC-III expression than mice fed a standard WTD (P<0.05, Student T-test). The effect of Compound A is more potent than the effect of reference Compound 12, an EPA derivative prepared according to Example 20 of WO2010/008299 having the following structure:

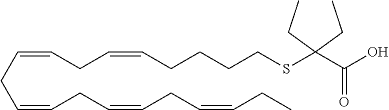 [SMILES: CC/C=C\C/C=C\C/C=C\C/C=C\C/C=C\CCCCSC(CC)(CC)C(=O)O]

### Reference Compound 12

In addition, the ability of both compounds to reduce plasma TG was measured. Both compounds reduced TG levels with 69% compared to control. This confirms that there is no direct correlation between the observed apoC-III reduction and TG lowering-effect.

### Example 25 Clinical Studies

The apoC-III reducing properties of Compound A have been demonstrated in two 12-week studies and one 4-week study in patients with dyslipidemia. All three studies demonstrated clinically and statistically significant reductions in apoC-III with Compound A treatment.

### Example 25A Population having Sever Hypertriglyceridemia

This study investigated patients with fasting plasma triglyceride levels above 500 mg/dL The primary objective of this study was to evaluate the efficacy of Compound A (Example 2) 600 mg once daily (QD) orally by assessment of the percentage change in triglycerides (TG) from baseline after 12 weeks of treatment. One of the secondary objectives was to evaluate the impact of Compound A on plasma levels of apoC-III.

This Phase II, multicenter, proof of concept study consisted of a 6- to 8-week screening period (which included a 4- or 6-week diet and lifestyle stabilization/washout period and a 2-week TG qualifying period), and a 12-week, double-blind, randomized, parallel group, placebo-controlled treatment period.

After confirmation of qualifying fasting TG values, eligible subjects entered the 12-week, randomized, double-blind treatment period. At Visit 4 (Week 0), subjects were randomly assigned in a 1:1 ratio to 1 of the following treatment groups: Compound A 600 mg QD or placebo QD.

Approximately 43 subjects per treatment group (approximately 86 subjects total) were to be randomized in this study. Stratification was by baseline TG level (≤700 mg/dL or >700 mg/dL), statin use at randomization, and gender.

The population for this study was men and women (women of childbearing potential were required to use adequate methods to avoid pregnancy) between the ages of 18 to 79 years of age, inclusive. Subjects on stable lipid-lowering statin therapy and subjects not on non-statin therapy were eligible to enroll in the study. Subjects were required to have an average fasting TG level ≥500 mg/dL, and ≤1500 mg/dL from Visit 2 and Visit 3 values or Visit 3 and Visit 3.1 values prior to randomization.

The intent-to-Treat (ITT) Population consisted of all randomized subjects who took at least 1 dose of investigational product, had a baseline efficacy measurement, and had at least 1 post-randomization efficacy measurement. The ITT Population was the primary analysis population, All efficacy analyses were performed on the ITT Population.

Summary statistics (n, mean, standard deviation [SD], median, minimum, and maximum) for the baseline and post-baseline measurements, the percent changes, or changes from baseline were presented by treatment group and by visit for all efficacy variables analyzed.

The primary efficacy analysis was performed using an analysis of covariance (ANCOVA) model with treatment, gender, and the use of statin therapy at randomization as factors and baseline TG value as a covariate. The least-squares means, standard errors, and 2-tailed 95% confidence intervals (CIs) for each treatment group and for the comparison between Compound A and placebo were provided.

An ANCOVA model was used for the analysis of secondary efficacy variables with treatment, gender, and the use of statin therapy at randomization as factors and the baseline value of the respective efficacy variable as a covariate.

The population recruited for the current study included men (69.0%) and women (31.0%) with a mean age of 52.5 years. approximately 21% of subjects in both treatment groups received statin therapy through the study. All other non-statin lipid-altering medications were discontinued at screening. Mean compliance to study medication during the study was 96.5% for the placebo group and 99.9% for the Compound A 600 mg group.

In the ITT Population, the least-squares (LS) mean percent change in apoC-III was −38.0% (−47.5, −28.5) vs baseline and −34.7% (−46.5, −22.8) versus placebo.

### Example 25B Population having Mixed Dyslipidemia

This study investigated patients with fasting plasma TG levels between 200 and 499 mg/dL and non-high density lipoprotein cholesterol (non-HDL-C) above 130 mg/dL already receiving treatment with statins. The primary objective of this study was to evaluate the efficacy of Compound A (Example 2) 600 mg QD orally by assessment of the percentage change in triglycerides non-HDL-C from baseline after 12. weeks of treatment. One of the secondary objectives was to evaluate the impact of Compound A on plasma levels of apoC-III.

This Phase II, multicenter, proof of concept study consisted of a 6- to 8-week screening period (which included a 4- or 6-week diet and lifestyle stabilization/washout period and a 2-week TG and non-HDL-C qualifying period), and a 12-week, double-blind, randomized, parallel group, placebo-controlled treatment period.

After confirmation of qualifying fasting TG and non-HDL-C values, eligible subjects entered the 12-week, randomized, double-blind treatment period. At Visit 4 (Week 0), subjects were randomly assigned in a 1:1 ratio to 1 of the following treatment groups: Compound A 600 mg QD or placebo QD.

The population for this study was men and women (women of childbearing potential were required to use adequate methods to avoid pregnancy) between the ages of 18 to 79 years of age, inclusive. Subjects on stable lipid-lowering statin therapy and subjects not on non-statin lipid-lowering therapy were eligible to enroll in the study. Subjects were required to have an average fasting TG level between 200 and 499 mg/L and non-HDL-C values above 130 mg/dL from Visit 2 and Visit 3 values or Visit 3 and Visit 3.1 values prior to randomization.

The Intent-to-Treat (ITT) Population consisted of all randomized subjects who took at least 1 dose of investigational product, had a baseline efficacy measurement, and had at least 1 post-randomization efficacy measurement. The ITT Population was the primary analysis population. All efficacy analyses were performed on the ITT Population.

Summary statistics (n, mean, standard deviation [SD], median, minimum, and maximum) for the baseline and post-baseline measurements, the percent changes, or changes from baseline were presented by treatment group and by visit for all efficacy variables analyzed.

The primary efficacy analysis was performed using an ANCOVA model with randomization as factor and baseline non-HDL-C value as a covariate. The least-squares means, standard errors, and 2-tailed 95% CIs for each treatment group and for the comparison between Compound A and placebo were provided.

The primary efficacy analysis was based on the 12-week completer population.

The population recruited for the current study included men (58.4%) and women (46.1%) with a mean age of 58.3 years. All subjects were required to be on statin therapy (with or without ezetimibe) during the study. All other non-statin lipid-altering medications were discontinued at screening. Mean compliance to study medication during the study was 97.2% for the placebo group and 95.3% for the Compound A group.

The baseline mean non-HDL-C level for the study population was 165.9 mg/dL; the baseline median TG level was 262.0 mg/dL.

In the 12-week completer population, the LS mean percent change in ApoC-III was −32.5% (−38.4, −26.6) vs baseline and −20.8% (−28.8, −12.7) vs placebo.

Example 25A refers to studies in patients with very high triglycerides (TG 500-2000 mg/dl). Example 25B refers to studies in statin stable patients with mixed dyslipidemia and persistent hypertriglyceridemia (TG 200-499 mg/dl). The studies included in each section are similar in design, with comparable patient populations.

### Example 25C Population having Hypercholesterolemia

This study investigated subjects with fasting LDL-C of at least 2.5 mmol (˜97 mg/dl). The objective of the study was to determine the pharmacodynamics and lipid lowering effects of Compound A (Example 2) following 4 weeks of treatment in male, hypercholesterolemic subjects withdrawn from stable statin therapy.

The population for this study consisted of men between 18 and 65 years of any ethnic origin and with a BMI between 18.0 and 35.0 kg/m².

This Phase Ib study consisted of a 4-5 week screening period, and a 4 week double-blind, randomized, placebo-controlled treatment period.

All subjects had to be on lipid-lowering statin therapy for at least 3 months prior to the first screening visit, and at stable statin dose for at least 4 weeks prior to the first screening vist.

Statin treatment was withdrawn at the first screening visit, and remained withdrawn for the entire screening period. Following withdrawal of statin medication for at least 21 days subject had to have an LDL-C of at least 2.5 mmol/l (˜97 mmol/l) at the secondary screening visit and an increase in LDL-C of at least 20% between the first screening visit and the secondary screening visit prior to randomization.

After confirmation of qualifying fasting LDL-C, eligible subjects entered a 4-week double blind, randomized, placebo-controlled treatment period. Subjects were randomly assigned in a 3:1 ratio to one of the following treatment groups: Compound A 600 mg QD (N=18) or placebo QD (N=6).

Blood lipids were measured at the end of the screening period and after 4 weeks of treatment, Exploratory pharmacodynamic measurements included LDL-C, VLDL-C, TC, TO, HDL-C, Non-HDL-C, and Apo B. The impact of Compound A on Apo C-III was also measured.

Summary statistics for baseline is given as mean with coefficient of variance. The mean changes from baseline with 95% confidence intervals were presented by treatment group for efficacy variables analyzed.

Analyses were performed using analysis of covariance (ANCOVA) model on changes from baseline with baseline included as covariate.

The population recruited for the current study included white males (100%) with a mean age of 55 years, mean weight of 85 kg, and mean BMI of 27.9 kg/m².

The mean percent change in Apo C-III after treatment with Compound A was −42% vs baseline. This change was statistically significant.

### Example 26 Comparative Reductions in apoC-III Achieved by EPA/DHA Versus Compound A

### (a) Effects of EPA/DHA Formulations Versus Compound A on Plasma apoC-III and Other Lipid Parameters in Subjects with Severe HTG.

### The MARINE Trial

In a double blind, randomized, placebo controlled study the effect of eicosapentaenoic acid ethyl ester (>96% by weight of the concentrate) (Vascepa) apoC-III was investigated in 229 patients with fasting plasma TG of 500-2000 mg/dl. Vascepa 4 g/day for 12 weeks reduced median apoC-III levels from 25.6 mg/dl to 19.7 mg/dl, corresponding to a median change from baseline of −10.1% [Journal of Clinical Lipidology 2014; 8(3): 313-314, Icosapent Ethyl (eicosapentaenoic acid ethyl ester): Effects on Apolipoprotein in patients from the MARINE and ANCHOR studies.] (Table 1).

### The EVOLVE Trial

In a double blind, randomized, placebo controlled study the effect of a combination of EPA and DHA as free fatty acids (55% by weight of EPA and 20% by weight of DHA) (Epanova) on apoC-III was investigated in 399 patients with fasting plasma TG of 500-2000 mg/dL. Epanova 4 g/day for 12 weeks resulted in a median apoC-III change from baseline of −15% [Circulation 2012; 126: A9030, Abstract 19030: Apolipoprotein C-III is Significantly Reduced by Prescription Omega-3 Free Fatty Acids (Epanova) in Patients with Severe Hypertriglyceridemia and Changes Correlate with Increases in LDL-C: A Sub-analysis of the EVOLVE trial](Table 1).

TABLE 1
Effect of treatment with omega-3 prescription pharmaceuticals and
Compound A in subjects with TG >500 mg/dl. Values are median
% changes from baseline.

| | | ApoC- | Non- | VLDL- | LDL- | |
|---|---|---|---|---|---|---|
| | TG | III | HDL-C | C | C | HDL-C |
| Compound A | −51 | −41 | −8 | −51 | 43 | 24 |
| Vascepa (omega-3) | −26.6 | −10.1 | −7.7 | −25.2 | −4.5 | −3.5 |
| Epanova (omega-3) | −30.9 | −15.0 | −9.6 | −33.0 | 19.4 | 5.8 |

### (b) Effects of EPA/DHA Formulations Versus Compound A on Plasma ApoC-III and Other Lipid Parameters in Statin Stable Subjects with Mixed Dyslipidemia and Persistent Hyperglyceridemia

### The ANCHOR Trial

In a double blind, randomized, placebo controlled study the effect of eicosapentaenoic acid ethyl ester (Vascepa) on apoC-III was investigated in 702 statin stable patients with mixed dyslipidemia and persistent hypertriglyceridemia with fasting plasma TG of 200-499 mg/l. Vascepa 4 g/day for 12 weeks reduced median apoC-III levels from 15.2 mg/dl to 13.7 mg/dl, corresponding to a median change from baseline of −9.4% [Journal of Clinical Lipidology 2015, in press, http://dx.dol.org/10.1016/j.jacl.2014.11.003, Effects of icosapent ethyl on lipoprotein particle concentration and size in statin-treated patients with persistent high triglycerides (the ANCHOR study)] (Table 2).

### The ESPRIT Trial

In a double blind, randomized, placebo controlled study the effect of a combination of EPA and DHA as free fatty acids (Epanova) apoC-III was investigated in 647 statin stable patients with mixed dyslipidemia and persistent hypertriglyceridemia with fasting plasma TG of 200-499 mg/dl. Epanova 4 g/day for 12 weeks resulted in a mean apoC-III change from baseline of −13.1% [JACC 2013; 61: E1468, A highly bioavailable omega-3 fatty acid reduces non-high density lipoprotein cholesterol in high-risk patients treated with a statin and residual hypertriglyceridemia (ESPRIT trial)] (Table 2).

### The COMBOS Trial

In a double blind randomized study the effect of a combination of EPA and DHA ethyl esters (46.5% by weight of EPA EE and 37.5% by weight of DHA EE) (Lovaza) on apoC-III was investigated in 256 statin stable patients with mixed dyslipidemia and persistent hypertriglyceridemia with fasting plasma TG of 200-400 mg/dl. Lovaza 4 g/day for weeks resulted in a median apoC-III change from baseline of −7.8% [Clinical Therapeutics 2007; 29(7): 1354-1367, Efficacy and tolerability of adding prescription Omega-3 fatty acids 4 g/d to simvastatin 40 mg/d in hypertriglyceridemic patients: An 8-week, randomized, double-blind, placebo-controlled study] (Table 2).

TABLE 2
Effect of treatment with omega-3 prescription pharmaceuticals
and Compound A in subjects with mixed dyslipidemia with
persistent hypertriglyceridemia (TG = 200-499 mg/dl).
Values are median % changes from baseline*.

| | | ApoC- | Non- | Apo | VLDL- | |
|---|---|---|---|---|---|---|
| | TG | III | HDL-C | B | C | LDL-C |
| Compound A | −43 | −35 | −10 | −6 | −39 | 0 |
| Vascepa (omega-3) | −17.5 | −9.4 | −5.0 | −2.2 | −12.1 | 1.5 |
| Epanova (omega-3) | −20.6 | −13.1* | −6.9 | −2.1 | −21.5 | 1.3 |
| Lovaza (omega-3) | −29.5 | −7.8 | −9.0 | −4.2 | −27.5 | 0.7 |

*ApoC-III value for Epanova is mean % change from baseline

### Summary of Comparative Reductions in Plasma apoC-III with EPA/DHA Versus Compound A

Although head-to-head trials have not been completed, the comparable patient populations and study designs provide a reasonable benchmark from which to compare the efficacy of Compound A versus EPA/DHA in lowering plasma apoC-III. There are two notable differentiating factors between the naturally occurring omega-3 fatty acids and Compound A.

The first is the superior potency of Compound A, which achieved a median reductions in apoC-III of 35 and 41% in the mixed dyslipidemic and severe HTG patient populations respectively. This compares with apoC-III reductions of only 7.8-15% in the EPA/DHA studies.

The second differentiating factor is the low-dose of Compound A needed (600 mg QD) versus the 4 g dose in the EPA/DHA studies. On a gram for gram basis, this difference is even greater for Compound A and clearly demonstrates the potency of this molecule in reducing plasma apoC-III versus EPA/DHA, As previously mentioned, pre-clinical models suggest that the apoC-III lowering is independent of TG lowering (FIG. 1).

1. A method of reducing apolipoprotein C-III (apoC-III) mRNA or protein in a subject in need thereof, comprising administering to the subject a pharmaceutically effective amount of a compound of Formula (I): 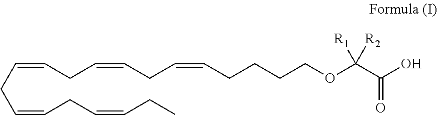
or a pharmaceutically acceptable salt or ester thereof,
wherein R₁ and R₂ are independently chosen from a hydrogen atom or linear, branched, and/or cyclic C₁-C₆ alkyl groups, with the proviso that R₁ and R₂ are not both hydrogen.

2. The method according to claim 1, wherein the compound is present in the form of an enantiomer, diastereomer, or mixture thereof.

3. The method according to claim 1, wherein R₁ and R₂ are chosen from a hydrogen atom, a methyl group, an ethyl group, a n-propyl group, and an isopropyl group.

4. The method according to claim 1, wherein R₁ and R₂ are chosen from a hydrogen atom, a methyl group, and an ethyl group.

5. The method according to claim 1, wherein one of R₁ and R₂ is a hydrogen atom and the other one of R₁ and R₂ is chosen from a C₁-C₃ alkyl group.

6. The method according to claim 1, wherein one of R₁ and R₂ is a hydrogen atom and the other one of R₁ R₂ is chosen from a methyl group or an ethyl group.

7. The method according to claim 1, wherein the ester is chosen from a glyceride, and a C₁-C₆ alkyl ester.

8. The method according to claim 1, wherein the ester is chosen from a triglyceride, a 1,2-diglyceride, a 1,3-diglyceride, a 1-monoglyceride, and 2-monoglyceride.

9. The method according to claim 1, wherein the ester is chosen from a methyl ester, an ethyl ester, an isopropyl ester, a n-butyl ester, and a tert-butyl ester.

10. The method according to claim 1, wherein the ester is selected from a methyl ester and an ethyl ester.

11. The method according to claim 2, wherein the compound is present in its R form.

12. The method according to claim 2, wherein the compound is present in its S form.

13. The method according to claim 2, where the compound is present in racemic form.

14. The method according to claim 1, wherein R₁ is hydrogen and R₂ is ethyl and the formula is

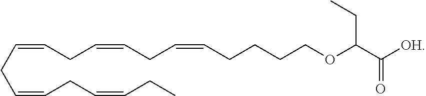 [SMILES: CC/C=C\C/C=C\C/C=C\C/C=C\C/C=C\CCCCOC(CC)C(=O)O]

15. The method according to claim 14, wherein the compound is present in its S and/or R form represented by the formulas:

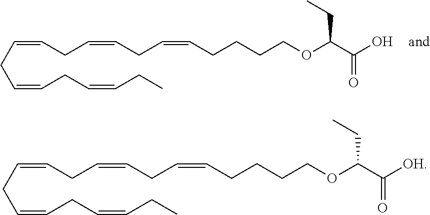 [SMILES: CC/C=C\C/C=C\C/C=C\C/C=C\C/C=C\CCCCO[C@@H](CC)C(=O)O ; CC/C=C\C/C=C\C/C=C\C/C=C\C/C=C\CCCCO[C@H](CC)C(=O)O]

16. The method according to claim 1, wherein the pharmaceutically effective amount of the compound of Formula (I) ranges from about 5 mg to about 2 g per dose.

17. The method according to claim 1, wherein the pharmaceutically effective amount of the compound of Formula (I) ranges from about 200 mg to about 800 mg per dose.

18. The method according to claim 1, wherein the pharmaceutically effective amount of the compound of Formula (I) is about 600 mg.

19. The method according to claim 1, wherein the subject is a human.

20. The method according to claim 1, wherein the compound is administered once daily.

21. The method according to claim 1, wherein the compound is formulated as a pharmaceutical composition for oral administration.

22. The method according to claim 21, wherein the pharmaceutical composition is in the form of a gelatin capsule or a tablet.

23. The method according to claim 22, wherein the pharmaceutical composition further comprises at least one binder, excipient, diluent, or any combinations thereof.

24. The method according to claim 22, wherein the pharmaceutical composition further comprises an antioxidant.

25. The method according to claim 24, wherein the antioxidant is chosen from tocopherol, BHA, and BHT, or a mixture thereof.

26. A method of reducing apoC-III in a subject in need thereof, the method comprising administering to the subject a pharmaceutically effective amount of 2-((5Z,8Z,11Z,14Z,17Z)-icosa-5,8,11,14,17-pentaen-1-yloxy)butanoic acid: 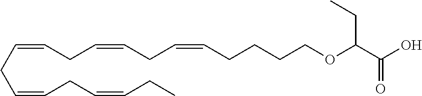 [SMILES: CC/C=C\C/C=C\C/C=C\C/C=C\C/C=C\CCCCOC(CC)C(=O)O]
or a pharmaceutically acceptable salt or ester thereof.

27. The method according to claim 26, wherein the pharmaceutically effective amount of 2-((5Z,8Z,11Z,14Z,17Z)-icosa-5,8,11,14,17-pentaen-1-yloxy)butanoic acid ranges from about 200 mg to about 800 mg per dose.

28. The method according to claim 27, wherein 2-((5Z,8Z,11Z,14Z,17Z)-icosa-5,8,11,14,17-pentaen-1-yloxy)butanoic acid is administered once daily.

29. Use of a pharmaceutically effective amount of a compound of Formula (I) 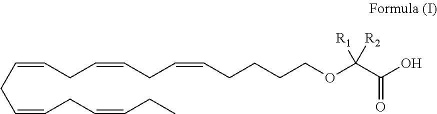
or a pharmaceutically acceptable salt or ester thereof,
wherein R₁ and R₂ are independently chosen from a hydrogen atom or linear, branched, and/or cyclic C₁-C₆ alkyl groups, with the proviso that R₁ and R₂ are not both hydrogen, in the manufacture of a medicament for reducing apolipoprotein C-III (apoC-III) mRNA or protein in a subject in need thereof.

30. The use according to claim 29, wherein the compound is present in the form of an enantiomer, diastereomer, or mixture thereof.

31. The use according to claim 29, wherein R₁ and R₂ are chosen from a hydrogen atom, a methyl group, an ethyl group, a n-propyl group, and an isopropyl group.

32. The use according to claim 29, wherein R₁ and R₂ are chosen from a hydrogen atom, a methyl group, and an ethyl group.

33. The use according to claim 29, wherein one of R₁ and R₂ is a hydrogen atom and the other one of R₁ and R₂ is chosen from a C₁-C₃ alkyl group.

34. The use according to claim 29, wherein one of R₁ and R₂ is a hydrogen atom and the other one of R₁ and R₂ is chosen from a methyl group or an ethyl group.

35. The use according to claim 29, wherein the ester is chosen from a glyceride, and a C₁-C₆ alkyl ester.

36. The use according to claim 29, wherein the ester is chosen from a triglyceride, a 1,2-diglyceride, a 1,3-diglyceride, a 1-monoglyceride, and 2-monoglyceride.

37. The use according to claim 29, wherein the ester is chosen from a methyl ester, an ethyl ester, an isopropyl ester, a tert-butyl ester, and a tert-butyl ester.

38. The use according to claim 29, wherein the ester is selected from a methyl ester and an ethyl ester.

39. The use according to claim 30, wherein the compound is present in its R form.

40. The use according to claim 30, wherein the compound is present in its S form.

41. The use according to claim 30, where the compound is present in racemic form.

42. The use according to claim 29, wherein R₁ is hydrogen and R₂ is ethyl and the formula is

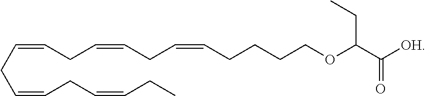 [SMILES: CC/C=C\C/C=C\C/C=C\C/C=C\C/C=C\CCCCOC(CC)C(=O)O]

43. The use according to claim 42, wherein the compound is present in its S and/or R form represented by the formulas:

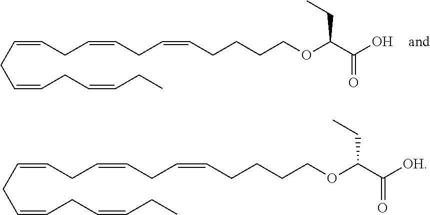 [SMILES: CC/C=C\C/C=C\C/C=C\C/C=C\C/C=C\CCCCO[C@@H](CC)C(=O)O ; CC/C=C\C/C=C\C/C=C\C/C=C\C/C=C\CCCCO[C@H](CC)C(=O)O]

44. The use according to claim 29, wherein the pharmaceutically effective amount of the compound of Formula (I) ranges from about 5 mg to about 2 g per dose.

45. The use according to claim 29, wherein the pharmaceutically effective amount of the compound of Formula (I) ranges from about 200 mg to about 800 mg per dose.

46. The use according to claim 29, wherein the pharmaceutically effective amount of the compound of Formula (I) is about 600 mg.

47. The use according to claim 29, wherein the subject is a human.

48. The use according to claim 29, wherein the compound is administered once daily.

49. The use according to claim 29, wherein the compound is formulated as a pharmaceutical composition for oral administration.

50. The use according to claim 49, wherein the pharmaceutical composition is in the form of a gelatin capsule or a tablet.

51. The use according to claim 50, wherein the pharmaceutical composition further comprises at least one binder, excipient, diluent, or any combinations thereof.

52. The use according to claim 50, wherein the pharmaceutical composition further comprises an antioxidant.

53. The use according to claim 52, wherein the antioxidant is chosen from tocopherol, BHA, and BHT, or mixtures thereof.

54. Use of a pharmaceutically effective amount of 2-((5Z,8Z,11Z,14Z,17Z)-icosa-5,8,11,14,17-pentaen-1-yloxy)butanoic acid: 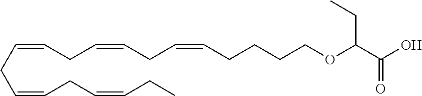 [SMILES: CC/C=C\C/C=C\C/C=C\C/C=C\C/C=C\CCCCOC(CC)C(=O)O]
or a pharmaceutically acceptable salt or ester thereof in the manufacture of a medicament for reducing apoC-III in a subject in need thereof.

55. The use according to claim 54, wherein the pharmaceutically-effective amount of 2-((5Z,8Z,11Z,14Z,17Z)-icosa-5,8,11,14,17-pentaen-1-yloxy)butanoic acid ranges from about 200 mg to about 800 mg per dose.

56. The use according to claim 55, wherein 2-((5Z,8Z,11Z,14Z,17Z)-icosa-5,8,11,14,17-pentaen-1-yloxy)butanoic acid is administered once daily.

57. The method according to claim 1, wherein the subject is on statin therapy and has baseline fasting triglycerides of about 200 mg/dl to about 499 mg/dl.

58. The method according to claim 1, wherein the subject has baseline fasting triglycerides of about 200 mg/dl to about 499 mg/dl.

59. The method according to claim 57 or 58, wherein the apoC-III level is reduced by at least about 20%.

60. The method according to claim 57 or 58, wherein the apoC-III level is reduced by at least about 35%.

61. The method according to claim 1, wherein the subject is on statin therapy and has fasting baseline triglycerides of above 500 mg/dl.

62. The method according to claim 1, wherein the subject has fasting baseline triglycerides of above 500 mg/dl.

63. The method according to claim 61 or 62, wherein the apoC-III level is reduced by at least about 25%.

64. The method according to claim 61 or 62, wherein the apoC-III level is reduced by at least about 40%.

65. The method according to claim 1, wherein the subject has fasting LDL-cholesterol of at least 2.5 mmol/L (˜97 mg/dl).

66. The method according to claim 65, wherein the apoC-III level is reduced by at least about 25%.

67. The method according to claim 65, wherein the apoC-III level is reduced by at least about 40%.

68. A method for reducing apoC-III in a subject in need thereof, comprising administering to the subject a pharmaceutically effective amount of a dyslipidemic agent and a compound of Formula (I): 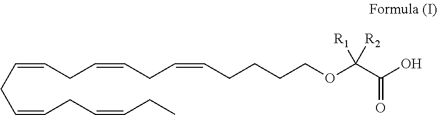
or a pharmaceutically acceptable salt or ester thereof,
wherein R₁ and R₂ are independently chosen from a hydrogen atom or linear, branched, and/or cyclic C₁-C₆ alkyl groups, with the proviso that R₁ and R₂ are not both hydrogen.

69. The method of claim 68, wherein the dyslipidemic agent is a statin.

70. The use according to claim 29, wherein the subject is on statin therapy and has baseline fasting triglycerides of about 200 mg/dl to about 499 mg/dl.

71. The use according to claim 29, wherein the subject has baseline fasting triglycerides of about 200 mg/dl to about 499 mg/dl.

72. The use according to claim 70 or 71, wherein the apoC-III level is reduced by at least about 20%.

73. The use according to claim 70 or 71, wherein the apoC-III level is reduced by at least about 35%.

74. The use according to claim 29, wherein the subject is on statin therapy and has fasting baseline triglycerides of above 500 mg/dl.

75. The use according to claim 29, wherein the subject has fasting baseline triglycerides of above 500 mg/dl.

76. The use according to claims 74 and 75, wherein the apoC-III level is reduced by at least about 25%.

77. The use according to claims 74 and 75, wherein the apoC-III level is reduced by at least about 40%.

78. The use according to claim 29, wherein the subject has fasting LDL-cholesterol of at least 2.5 mmol/L (˜97 mg/dl).

79. The use according to claim 78, wherein the apoC-III level is reduced by at least about 25%.

80. The use according to claim 78, wherein the apoC-III level is reduced by at least about 40%.

81. The method according to claim 1, wherein the subject in need has a disease or condition chosen from severe hypertriglyceridemia, mixed dyslipidemia, and hypercholesterolemia.

82. The use according to claim 29, wherein the subject in need has a disease or condition chosen from severe hypertriglyceridemia, mixed dyslipidemia, and hypercholesterolemia.
